(19) United States
(12) Patent Application Publication
(10) Pub. No.: US 2025/0235421 A1
(43) Pub. Date: Jul. 24, 2025

(54) VERY-LONG-CHAIN POLYUNSATURATED FATTY ACIDS (VLCPUFA) FOR IMPROVING RETINA/COGNITIVE FUNCTIONS AND ATHEROSCLEROSIS

(71) Applicant: Nissui Corporation, Tokyo (JP)

(72) Inventors: Zhi-Hong Yang, North Bethesda, MD (US); Alan Thomas Remaley, Bethesda, MD (US); Krishna Vamsi S. Rojulpote, King of Prussia, PA (US); Jingrong Tang, Great Falls, VA (US); Isao Yamazaki, Yokohama, Kanagawa (JP); Hideaki Yamaguchi, Hachioji, Tokyo (JP); Seizo Sato, Hachioji, Tokyo (JP)

(21) Appl. No.: 18/881,578

(22) PCT Filed: Jul. 7, 2023
(86) PCT No.: PCT/US2023/027076

§ 371 Date: Jan. 6, 2025

Related U.S. Application Data
(60) Provisional application No. 63/359,222, filed on Jul. 8, 2022.

(51) Int. Cl. A61K 31/202; A61P 1/16; A61P 3/04; A61P 3/06; A61P 3/08; A61P 9/10; A61P 25/28; A61P 27/02

(52) U.S. Cl. CPC A61K 31/202; A61P 1/16; A61P 3/04; A61P 3/06; A61P 3/08; A61P 9/10; A61P 25/28; A61P 27/02

(57) ABSTRACT

The present invention is directed to methods for treating macular degeneration, atherosclerosis, fatty liver, obesity, and cognitive ability, and more specifically to methods for treating macular degeneration, atherosclerosis, fatty liver, obesity, and cognitive ability using very long chain polyunsaturated fatty acids having 24 to 40 carbon atoms.

### CROSS-REFERENCE TO RELATED APPLICATIONS

This application claims priority to/from and the benefit of U.S. Provisional Application No. 63/359,222 filed on Jul. 8, 2022, which is incorporated herein by reference in its entirety.

### STATEMENT OF GOVERNMENT SUPPORT

This invention was made in part with Government support from the US Department of Health and Human Services, National Institutes of Health. The US government has certain rights in this invention.

### BACKGROUND OF THE INVENTION

### 1. Field of the Invention

The present invention is directed to methods for treating macular degeneration, atherosclerosis, fatty liver, obesity, and cognitive ability, and more specifically to methods for treating macular degeneration, atherosclerosis, fatty liver, obesity, and cognitive ability using very long chain polyunsaturated fatty acids having 24 to 40 carbon atoms.

### 2. Brief Description of the Related Art

The CDC reports an estimated 96 million adults aged 18 years or older had prediabetes in 2019. In 2018, a total of 8.25 million hospital discharges were reported with diabetes as any listed diagnosis among US adults aged 18 years or older. These discharges included 1.87 million for major cardiovascular diseases (74.4 per 1,000 adults with diabetes), including 440,000 for ischemic heart disease (17.5 per 1,000 adults with diabetes). There are more than 46 million older adults aged 65 and older living in the U.S., and by 2050, that number is expected to grow to almost 90 million. The global population aged 60 years and over will increase from 1 billion in 2020 to 1.4 billion by 2050.

Common health conditions related to ageing include diabetes, atherosclerosis, vision- and hearing-related problems (including cataracts and age-related macular degeneration), cognitive function decline, obesity, etc. According to the CDC, 50 percent of adults age 65 and older have prediabetes and 25 percent have diabetes. These conditions are caused by multiple factors, including insulin resistance, usually as a result of obesity and inactivity; reduced insulin production from the pancreas; and loss of muscle mass. People with prediabetes have a greater chance of developing type 2 diabetes and having a heart attack or stroke.

Aging is also the dominant risk factor for clinically significant atherosclerotic lesion formation. In addition, age-related macular degeneration is the most common cause of severe loss of eyesight among people 50 and older. However, these health problems can also significantly impact younger age groups of the population.

Very long chain fatty acids (VLCFAs) have structurally unusual long hydrocarbon chains (C24-C40). While present in extremely small quantities, VLCFAs are found in a number of species and organs (e.g., testes, retinas, brain and sperm), and they are essential lipids that play important roles in certain biological systems that cannot be fulfilled by the more common shorter chain C16-Cis fatty acids. Because of their very long chain structure, some VLCFAs are able to span and reside within both leaflets of the lipid bilayer, thereby giving stability to highly curved cellular membranes, such as those which surround nuclear pore complexes. In photoreceptors, the VLC-polyunsaturated FAs (VLCPUFA) are known to be associated with rhodopsin and play a role in regulation of phototransduction cascades. Absence of these VLCPUFAs appears to contribute to macular degeneration in autosomal dominant Stargardt macular dystrophy (STGD3).

The health benefits of fish diet and fish oil have attracted great scientific attention since 1970s when epidemiological studies proposed heart benefits of fish oil-derived n-3 polyunsaturated fatty acids (PUFAs), namely eicosapentaenoic acid (EPA; 20:5n-3) and docosahexaenoic acid (DHA; 22:6n-3). Retina n-3 PUFA depletion in mammalian models largely interfered with normal neurological function and visual signaling pathways that lead to impaired vision, and epidemiological studies have shown that a diet high in n-3 PUFA and oily fish was associated with a lower risk for advanced AMD. However, supplementation of EPA+DHA did not show benefit for AMD progression in several large, randomized, controlled trials.

Many breakthrough drugs and treatments have been developed in medical practice that have helped treat the conditions described above. However, there is a continued need to address these problems and find new methods and compositions to for their treatment. The present invention is believed to be an answer to that need.

### SUMMARY OF THE INVENTION

In one aspect, the present invention is directed to a method of treating or decreasing the risk of developing a condition responsive to age-related macular degeneration in a subject, comprising administering to said subject a therapeutically effective amount of a 24-carbon to 28-carbon very long chain polyunsaturated fatty acid (VLCPUFA).

In another aspect, the present invention is directed to a method for treating a disease or condition selected from hyperlipidemia, hypercholesterolemia or hypertriglyceridemia or a combination thereof, comprising administering to a subject in need of treatment a therapeutically effective amount of a 24-carbon to 40-carbon very long chain polyunsaturated fatty acid (VLCPUFA).

In another aspect, the present invention is directed to a method for treating hepatic steatosis, comprising administering to a subject in need thereof a therapeutically effective amount of a 24-carbon to 40-carbon very long chain polyunsaturated fatty acid (VLCPUFA).

In another aspect, the present invention is directed to a method for treating an elevated plasma glucose concentration in a subject in need thereof, comprising administering to said subject an effective amount of a composition comprising a 24-carbon to 40-carbon very long chain polyunsaturated fatty acid (VLCPUFA).

In another aspect, the present invention is directed to a method of treating, or ameliorating one or more symptoms of, atherosclerosis in a subject, comprising administering to said subject a therapeutically effective amount of a composition comprising a 24-carbon to 40-carbon very long chain polyunsaturated fatty acid (VLCPUFA).

In another aspect, the present invention is directed to a method of treating, or ameliorating one or more symptoms of, an adipofascial disorder in a subject, comprising administering to said subject a therapeutically effective amount of a composition comprising a 24-carbon to 40-carbon very long chain polyunsaturated fatty acid (VLCPUFA).

In another aspect, the present invention is directed to a method of treating, or ameliorating one or more symptoms of, impaired cognitive function in a subject, comprising administering to said subject a therapeutically effective amount of a composition comprising a 24-carbon to 40-carbon very long chain polyunsaturated fatty acid (VLCPUFA).

In some of the aspects above, the very long chain polyunsaturated fatty acid is preferably C24:5 n-3, C26:6 n-3, or C28:8 n-3 or a mixture thereof.

### BRIEF DESCRIPTION OF THE DRAWINGS

FIG. 1 shows body weight change in C57BL/6J mice. Seven-month-old mice were fed a chow diet supplemented with 1% (w/w) or 5% VLCPUFA concentrate oil, or none (control) for 8 weeks. Data are expressed as mean±SE, n=8 per group.

FIG. 2 shows changes in retina VLCPUFA composition in C57BL/6J mice. Eight-week-old mice were gavaged VLCPUFA oil. Plasma levels (% of total plasma fatty acids) of (A) C24 VLCPUFAs, (B) C26 VLCPUFAs, (C) C28 VLCPUFAs, (D) C30 VLCPUFAs, (E) C32 VLCPUFAs, (F) C34 VLCPUFAs, (G) total n-3 and n-6 VLCPUFAs, (H) n-3/n-6 VLCPUFAs ratio at 0, 2, 4, 8 and 24 hr of gavage. Data are expressed as mean±SE, n=6 per group, *P<0.05 vs. 0 hr.

FIG. 3 shows that dietary VLCPUFA improved photopic electroretinographic responses in C57BL/6J mice. The electroretinography (ERG) test was used to measure the electrical response of the light-sensitive cells in the eyes. Eight-week-old C57BL/6J mice were daily oral gavaged VLCPUFA oil or vehicle (control) for 2 weeks. Amplitudes of scotopic ERG a- and b-waves (FIGS. 3A and B), and photopic ERG a- and b-waves (FIGS. 3C and D), are plotted versus flash intensity. Data are expressed as mean±SE, n=6 per group. *P<0.05, ***P<0.001 vs. control.

FIGS. 4A and B show that dietary VLCPUFA dose-dependently improved scotopic and photopic electroretinographic responses in ApoE-KO mice. Nine-month-old ApoE-KO mice were fed a chow diet supplemented with 1% (w/w) or 3% VLCPUFA oil, or none (ApoE-KO mice control) for 8 weeks. The age-matched C57BL/6J mice were used as wild-type mice control. Data are expressed as mean±SE, n=8 per group. *P<0.05, **P<0.01, ***P<0.001, ****P<0.0001 vs. WT mice control. #P<0.05, ##P<0.01, ###P<0.001 vs. ApoE-KO mice control.

FIG. 5 shows that dietary VLCPUFA improved photopic and scotopic visual acuity in C57BL/6J mice. Visual acuity test was used to assess visual discrimination and acuity. Eight-week-old mice were daily oral gavaged VLCPUFA oil or vehicle (control) for 2 weeks. Data are expressed as mean±SE, n=6 per group. *P<0.05, ***P<0.001 vs. control.

FIG. 6 shows that dietary VLCPUFA improved (reduced) plasma lipid and glucose metabolic biomarkers in C57BL/6J mice. (A) Plasma phospholipid and cholesterol FPLC profile from pooled plasma, (B) plasma triglycerides and cholesterol levels, and (C) plasma glucose and insulin levels. Seven-month-old mice were fed a chow diet supplemented with 1% (w/w) or 5% VLCPUFA oil, or none (control) for 8 weeks. Data are expressed as mean±SE, n=8 per group. **P<0.01 vs. control.

FIG. 7 shows that dietary VLCPUFA improved (reduced) plasma lipid and glucose metabolic biomarkers in ApoE-KO mice. (A) Plasma total cholesterol, free cholesterol, and triglycerides levels. (B) plasma cholesterol FPLC profile from pooled plasma, and (C) plasma glucose levels. Nine-month-old mice were fed a chow diet supplemented with 1% (w/w) or 3% VLCPUFA oil, or none (ApoE-KO mice control) for 8 weeks. The age-matched C57BL/6J mice were used as wild-type mice control. Data are expressed as mean±SE, n=8 per group. *P<0.05, **P<0.01, ***P<0.001, ****P<0.0001 vs. WT mice control. #P<0.05, ##P<0.01 vs. ApoE-KO mice control.

FIG. 8 shows that dietary VLCPUFA improved (reduced) hepatic lipid accumulation in C57BL/6J mice. (A) Hematoxylin-eosin (H & E) and Oil-Red O staining of liver sections from representative mice from each group; the original magnification is 100×. (B) Enzymatic measurement of hepatic triglycerides (TG), phospholipid (PL) and cholesterol (TC) contents. (C) LC-MS/MS analysis of free cholesterol, cholesterol esters, and triglycerides contents in liver. Seven-month-old mice were fed a chow diet supplemented with 1% (w/w) or 5% VLCPUFA concentrate, or none (control) for 8 weeks. Data are expressed as mean±SE, n=8 per group. *P<0.05, **P<0.01, ***P<0.001 vs. control.

FIG. 9 shows 2D representation of the Partial Least-Squares Discriminant Analysis (PLS-DA) of lipidomic composition in C57BL/6J mice. (A) Plasma, (B) liver, (C) brain, (D) eye, and (E) testis. Seven-month-old mice were fed a chow diet supplemented with 1% (w/w) or 5% VLCPUFA concentrate, or none (control) for 8 weeks. n=8 per group.

FIG. 10 shows heatmaps of lipidomic classes detected and changes due to VLCPUFA-rich diet in C57BL/6J mice. (A) Plasma, (B) liver, (C) eye, (D) brain, and (E) testis. Seven-month-old mice were fed a chow diet supplemented with 1% (w/w) or 5% VLCPUFA concentrate, or none (control) for 8 weeks. n=8 per group.

FIG. 11 shows that dietary VLCPUFA increased plasma levels of certain VLCPUFAs. Seven-month-old mice were fed a chow diet supplemented with 1% or 5% VLCPUFA concentrate, or none (control) for 8 weeks. Data are expressed as mean±SE, n=8 per group.

FIG. 12 shows that dietary VLCPUFA improved body composition of ApoE-KO mice. (A) Body weight change, and (B) body composition based on NMR analysis. Nine-month-old mice were fed a chow diet supplemented with 1% (w/w) or 3% VLCPUFA concentrate oil, or none (control) for 8 weeks. The age-matched C57BL/6J mice were used as wild-type mice control. Data are expressed as mean±SE, n=8 per group. *P<0.05, ****P<0.0001 vs. WT mice control.

FIG. 13 shows that dietary VLCPUFA improved (reduced) progression of atherosclerosis in ApoE-KO mice. (A) Representative enface Sudan IV staining of aorta, and (B) quantitative analysis of Sudan IV-positive plaque area of aorta. Nine-month-old mice were fed a chow diet supplemented with 1% (w/w) or 3% VLCPUFA concentrate oil, or none (control) for 8 weeks. The age-matched C57BL/6J mice were used as wild-type mice control. Data are expressed as mean±SE, n=8 per group. *P<0.05 vs. ApoE-KO mice control.

FIG. 14 shows that dietary VLCPUFA improved spatial learning and memory in ApoE-KO mice. (A) The average latency to the platform of female mice, and (B) area under the curve of latency (days 1-6). Morris water maze test was used to assess spatial learning and memory in mice. Nine-month-old mice were fed a chow diet supplemented with 1% (w/w) or 3% VLCPUFA concentrate oil, or none (control) for 8 weeks. The age-matched C57BL/6J mice were used as wild-type mice control. Data are expressed as mean±SE, n=8 per group. *P<0.05 vs. ApoE-KO mice control.

FIG. 15 shows that dietary VLCPUFA improved contextual learning in ApoE-KO mice. (A) Baseline % time freezing, (B) contextual % time freezing, (C) novel context % time freezing, and (D) auditory cue % time freezing. The contextual fear learning test was used to assesses the ability of mice to learn and remember an association between environmental cues and aversive experiences. Nine-month-old mice were fed a chow diet supplemented with 1% (w/w) or 3% VLCPUFA concentrate oil, or none (control) for 8 weeks. The age-matched C57BL/6J mice were used as wild-type mice control. Data are expressed as mean±SE, n=8 per group. *P<0.05 vs. respective control.

FIG. 16 shows that VLCPUFA exhibited dose-dependent agonist activity with (A) PPARα and (B) PPARγ in CHO cells. CHO cells were used as reporter cells in the assay that expresses a receptor hybrid in which the native N-terminal DNA-binding domain (DBD) has been replaced with that of the yeast Gal4 DBD. The reporter gene, firefly luciferase, is functionally linked to the Gal4 upstream activation sequence (UAS). Cells were treated with VLCPUFA in serum with BSA for 24 hr prior to performing the luciferase assay. Data are expressed as mean±SE. All treatment concentrations were performed in triplicate. **P<0.01, ****P<0.0001 vs. vehicle.

FIGS. 17A and 17B show effects of dietary VLCPUFA on liver histology in C57BL/6J mice. Seven-month-old mice were fed a chow diet supplemented with 1% or 5% VLCPUFA concentrate, or none (control) for 8 weeks. (A) H & E staining and (B) Oil-Red 0 staining of liver sections from mice fed each diet, n=8.

FIG. 18 shows effects of dietary VLCPUFA on kidney histology in C57BL/6J mice. Seven-month-old mice were fed a chow diet supplemented with 1% or 5% VLCPUFA concentrate, or none (control) for 8 weeks. H & E staining of kidney sections from mice fed each diet, n=8.

FIG. 19 shows effects of dietary VLCPUFA on spleen histology in C57BL/6J mice. Seven-month-old mice were fed a chow diet supplemented with 1% or 5% VLCPUFA concentrate, or none (control) for 8 weeks. H & E staining of spleen sections from mice fed each diet, n=8.

FIG. 20 shows effects of dietary VLCPUFA on heart histology in C57BL/6J mice. Seven-month-old mice were fed a chow diet supplemented with 1% or 5% VLCPUFA concentrate, or none (control) for 8 weeks. H & E staining of heart sections from mice fed each diet, n=8.

FIG. 21 shows effects of dietary VLCPUFA on skeletal muscle histology in C57BL/6J mice. Seven-month-old mice were fed a chow diet supplemented with 1% or 5% VLCPUFA concentrate, or none (control) for 8 weeks. H & E staining of skeletal muscle sections from mice fed each diet, n=8.

FIG. 22 shows effects of dietary VLCPUFA on small intestine histology in C57BL/6J mice. Seven-month-old mice were fed a chow diet supplemented with 1% or 5% VLCPUFA concentrate, or none (control) for 8 weeks. H & E staining of small intestine sections from mice fed each diet, n=8.

FIG. 23 shows effects of dietary VLCPUFA on brain histology in C57BL/6J mice. Seven-month-old mice were fed a chow diet supplemented with 1% or 5% VLCPUFA concentrate, or none (control) for 8 weeks. H & E staining of brain sections from mice fed each diet, n=8.

FIG. 24 shows effects of dietary VLCPUFA on testis histology in C57BL/6J mice. Seven-month-old mice were fed a chow diet supplemented with 1% or 5% VLCPUFA concentrate, or none (control) for 8 weeks. H & E staining of male testis sections from mice fed each diet, n=4.

### DETAILED DESCRIPTION OF THE INVENTION

### Chemical Description and Terminology

Compounds are described using standard nomenclature. Unless defined otherwise, all technical and scientific terms used herein have the same meaning as is commonly understood by one of skill in the art to which this invention belongs. Unless clearly contraindicated by the context, each compound name includes the free acid or free base form of the compound as well as all pharmaceutically acceptable salts of the compound.

The terms “a” and “an” do not denote a limitation of quantity, but rather denote the presence of at least one of the referenced items. The term “or” means “and/or”. The open-ended transitional phrase “comprising” encompasses the intermediate transitional phrase “consisting essentially of” and the close-ended phrase “consisting of”. Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the fatty acid synthesis method or the deuterated fatty acids disclosed herein. Unless defined otherwise, technical and scientific terms used herein have the same meaning as is commonly understood by one of skill in the art to which this disclosure belongs.

The terms “or/and” and “and/or” mean “either . . . or . . . , or both . . . and . . . ” when referring to two elements, and mean “either . . . , . . . or . . . , or any combination or all thereof” when referring to three or more elements. As an example, the phrase “A or/and B” means “either A or B, or both A and B”, and the phrase “A, B or/and C” means “either A, B or C, or any combination or all thereof”.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

A dash (“-”) that is not between two letters or symbols is used to indicate a point of attachment for a substituent.

“Halo” or “halogen” means fluorine, chlorine, bromine or iodine.

The term “compound” encompasses salts, solvates, hydrates, clathrates and polymorphs of that compound or a salt of that compound. A “solvate” of a compound comprises a stoichiometric or non-stoichiometric amount of a solvent (e.g., water, acetone or an alcohol [e.g., ethanol]) bound non-covalently to the compound. A “hydrate” of a compound comprises a stoichiometric or non-stoichiometric amount of water bound non-covalently to the compound. A “clathrate” of a compound contains molecules of a substance (e.g., a solvent) enclosed in a crystal structure of the compound. A “polymorph” of a compound is a crystalline form of the compound. The specific recitation of “salt”, “solvate”, “hydrate”, “clathrate” or “polymorph” with respect to a compound in certain instances of the disclosure shall not be interpreted as an intended omission of any of these forms in other instances of the disclosure where the term “compound” or the like is used without recitation of any of these forms.

“Pharmaceutical compositions” are compositions comprising at least one active agent, such as a VLCPUFA or a salt thereof, and at least one other substance, such as a carrier or excipient. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as hepatitis C.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol or acetonitrile are preferred, where practicable. Salts of the present compounds further include solvates and hydrates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17^{th} Ed., Mack Publishing Company, Easton, Pennsylvania, p. 1418 (1985).

The term “carrier” applied to pharmaceutical compositions/combinations of the invention refers to a diluent, excipient or vehicle with which an active compound is provided.

A “pharmaceutically acceptable excipient” means an excipient that is useful in preparing a pharmaceutical composition/combination that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

A “patient” is a subject in need of medical treatment. The subject or patient can be a mammalian or non-mammalian animal, such as a companion animal (e.g., a dog or cat) or a livestock animal (e.g. a bovine, swine, equine or sheep). In some embodiments, the subject or patient is a human. Medical treatment can include treatment of an existing condition (e.g., a disease or disorder), prophylactic or preventative treatment, or diagnostic treatment.

“Treatment” as used herein includes providing a VLCPUFA or a salt thereof, either as the only active agent or together with at least one additional active agent, sufficient to: (a) prevent a disease or a symptom of a disease from occurring in a patient who may be predisposed to the disease but has not yet been diagnosed as having it (e.g., including macular degeneration in patients having a genetic mutation that predisposes them to macular degeneration such as the autosomal dominant mutation associated with Stargardt muscular dystrophy); (b) inhibit the disease, i.e., arrest its development; and (c) relieve the disease, i.e., cause regression of the disease. “Treating” and “treatment” also include providing a therapeutically effective amount of a VLCPUFA or a salt thereof, as the only active agent or together with at least one additional active agent, to a patient having or susceptible to a condition in which very long chain fatty acids are known to play a role.

“Preventing” a disease or disorder means effecting a statistically significant decrease in the likelihood of developing a disease or disorder in a patient at risk of developing the disease or disorder, or effecting a statistically significant delay in the onset of symptoms, or reducing the severity of symptoms in a patient at risk of developing the disease or disorder.

A “therapeutically effective amount” of a pharmaceutical composition/combination of this invention means an amount effective, when administered to a patient, to provide a therapeutic benefit such as an amelioration of symptoms, e.g., an amount effective to decrease the symptoms.

As used herein, the term “administering” means providing a pharmaceutical agent or composition to a subject, and includes, but is not limited to, administering by a medical professional and self-administering.

Fatty acids discussed herein are identified using the following conventional numbering system:

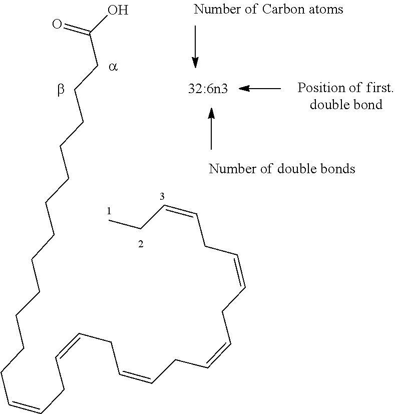 [SMILES: CC/C=C\C/C=C\C/C=C\C/C=C\C/C=C\C/C=C\CCCCCCCCCCCCC(=O)O]

In different aspects, methods of treatment employing compositions of very long chain polyunsaturated fatty acids (VLCPUFA) for treating or ameliorating various clinical conditions including atherosclerosis, impaired cognitive function, elevated plasma glucose concentrations, age-related macular degeneration and other conditions are described.

In an aspect, the very long chain polyunsaturated fatty acids (VLCPUFA) have between 24 and 40 carbon chain length. In an aspect, the very long chain polyunsaturated fatty acids (VLCPUFA) have one or more double bonds of cis or trans geometry. In an aspect, fatty acids described herein are identified by a letter-number name such as C24:5 n-3. The letter “C” denotes carbon, the number before the colon specifies the number of carbon atoms, and the number after the colon specifies the number of double bonds. The position of the terminal double bond is denoted in the form n-x, where n represents the locant of the methyl end of the molecule and x refers to the locant of the double bond closest to the methyl end of the molecule, or the number of carbon atoms from the methyl end of the molecule to the nearest double bond:

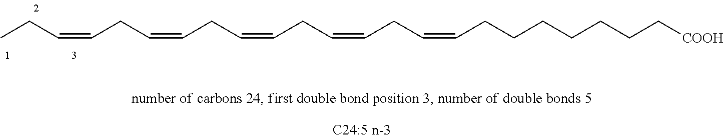 [SMILES: CC/C=C\C/C=C\C/C=C\C/C=C\C/C=C\CCCCCCCC(=O)O]

### Pharmaceutical Compositions and Methods of Treatment

Provided herein are pharmaceutical compositions comprising at least one very long chain polyunsaturated fatty acid (VLCPUFA) together with a pharmaceutically acceptable carrier or excipient for use in treating the medical conditions described herein. A pharmaceutical composition can comprise an individual VLCPUFA or a mixture of VLCPUFAs. Likewise, a medical condition described herein can be treated by administering an individual VLCPUFA or a mixture of VLCPUFAs. In some embodiments, the mixture of VLCPUFAs is a mixture of VLCPUFAs derived from fish oil, such as fish oil-derived VLCPUFA concentrate oil that can also contain PUFAs having fewer than 24 carbon atoms. In further embodiments, the VLCPUFA is C24:5 n-3, C26:6 n-3 or C28:8 n-3, or any combination or all thereof.

In addition, a pharmaceutical composition can comprise, and a medical condition described herein can be treated by administering, one or more polyunsaturated fatty acids (PUFAs) having fewer than 24 carbon atoms, such as 20 or 22 carbon atoms. In some embodiments, the one or more PUFAs are a mixture of PUFAs derived from fish oil. In further embodiments, the one or more PUFAs are C20:2 n-6, C20:4 n-6, C22:4 n-6 or C22:6 n-3, or any combination or all thereof.

The pharmaceutical composition may have any suitable form, and may be a tablet, capsule, lyophilized solid, solution, suspension, or a combination thereof. The pharmaceutical composition may be, e.g., an injectable (e.g., intravenous, subcutaneous or intramuscular), topical (e.g., ophthalmic or transdermal), or oral dosage form. The pharmaceutical composition may be a dosage form intended for parenteral (e.g., intravenous, subcutaneous or intramuscular) administration, such as a pre-prepared solution in a vial or syringe, a lyophilized solid needing reconstitution before administration, or a reconstituted solution of the lyophilized solid. The pharmaceutical composition can be an ocular formulation, such as a liquid topical formulation. The pharmaceutical composition may be an oral dosage form in the form of, e.g., a tablet or capsule. In a preferred embodiment, the VLCFA is formulated into any oral dosage form including solid, semi-solid, liquid, powder, sachet and the like. Solid oral dosage forms can include, for example, a tablet, a capsule (hard or soft), or subunits, and the like. “Subunit” includes a minitablet, a bead, a spheroid, a microsphere, a seed, a pellet, a caplet, a microcapsule, a granule, a particle, and the like that can provide an oral dosage form alone or when combined with other subunits. Exemplary semi-solid or liquid dosage forms include a suspension, a solution, an emulsion, and the like. Solid oral dosage forms can also include orally dissolving/disintegrating dosage (ODT) forms. Exemplary ODTs include orally dissolving/disintegrating tablets, orally dissolving films and dosage forms intended for sublingual/lingual/buccal delivery such as fast dissolving/disintegrating sublingual tablets and films.

The oral dosage forms used in the methods and compositions can be formulated for a specific type of release including immediate-release, controlled-release, sustained-release, or extended-release.

The disclosure includes methods and compositions in which one or more compounds are an admixture or otherwise combined with one or more compounds and may be in the presence or absence of commonly used excipients (or “pharmaceutically acceptable carriers”), such as, but not limited to: i) diluents and carriers such as starch, mannitol, lactose, dextrose, sucrose, sorbitol, cellulose, or the like; ii) binders such as starch paste, gelatin, magnesium aluminum silicate, methylcellulose, alginates, gelatin, sodium carboxymethyl-cellulose, polyvinylpyrrolidone or the like; iii) lubricants such as stearic acid, talcum, silica, polyethylene glycol, polypropylene glycol or the like; iv) absorbents, colorants, sweeteners or the like; v) disintegrants (e.g., calcium carbonate and sodium bicarbonate) such as effervescent mixtures or the like; vi) excipients (e.g. cyclodextrins or the like); vii) surface active agents (e.g., cetyl alcohol and glycerol monostearate), adsorptive carriers (e.g., kaolin and bentonite), emulsifiers or the like. Examples of carriers include, without limitation, any liquids, liquid crystals, solids and semi-solids, such as water and saline, gels, creams, salves, solvents, diluents, fluid ointment bases, ointments, pastes, implants, liposomes, micelles, giant micelles, and the like, which are suitable for use in the compositions. The disclosure of this paragraph also applies to pharmaceutical compositions comprising one or more VLCPUFAs, optionally in combination with one or more additional therapeutic agents.

The disclosure includes ophthalmic compositions. The disclosed compositions can be emulsions, solutions, suspensions, gels, ointments, occlusive films, or sustained-release films, and they can be preserved or non-preserved formulations. The compositions can be formulated as eye drops, creams, ointments, and films that can be applied to an eye. The formulations can be administered to the eye, the upper eye lid, the lower eye lid, or a combination thereof.

Ophthalmic compositions can include polymeric emulsifiers, such as castor oil, squalene, isostearate, and isopropyl myristate; lipophilic components, such as mineral oil, silicone oil, and caprylic/capric triglycerides; and/or alcohols, such as cetyl alcohols and stearyl alcohols. Ophthalmic compositions of the disclosure may also contain diethylene glycol monoethyl ether, propylene glycol, and/or dipropylene glycol; co-solvents such as dimethyl ether, diethylene glycol, and dipropylene glycol; and/or buffers and pH-modifying agents such as sodium citrate dihydrate, boric acid, monosodium phosphate, dibasic heptahydrate, and sodium phosphate monobasic monohydrate. Ophthalmic compositions can contain cyclodextrin, hydroxypropyl-beta-cyclodextrin, hydroxyethyl cellulose, PEG 300, PEG 400, povidone, glycerin, propylene glycol, or hydroxypropyl methyl cellulose, or any combination thereof. Suitable preservatives include, e.g., benzalkonium chloride. The ophthalmic composition can contain a plasticizer or a film former. Ophthalmic compositions typically contain a vehicle such as water or an isotonic solvent system (e.g., phosphate-buffered saline), in which the active compound (e.g., a VLCPUFA) has a concentration of about 0.01-90%, 0.1-50%, 0.1-30%, 0.5-20% or 1-10% wt/vol.

The disclosure includes methods and compositions prepared using conventional mixing, granulating, or coating methods and may contain 0.01 to 90% of the active ingredients. The resulting compositions (formulations) may be presented in unit dosage form and may be prepared by methods known in the art of pharmacy. All methodology includes the act of bringing the active ingredient(s) into association with the carrier which constitutes one or more ingredients. Therefore, compositions (formulations) may be prepared by blending active ingredient(s) with, e.g., a liquid carrier and/or a finely divided solid carrier, and then, if needed, shaping the product into a desired formulation.

Certain compositions and methods of the disclosure contain a compound described herein (e.g., a VLCPUFA) from about 90% to about 80% by weight, from about 80% to about 70% by weight, from about 70% to about 60% by weight, from about 60% to about 50% by weight, from about 50% to about 40% by weight, from about 40% to about 30% by weight, from about 30% to 20% by weight, from about 20% to about 10% by weight, from about 10% to about 4% by weight, from about 4% to about 2% by weight, from about 2% to about 1% by weight, or from about 1% to about 0.01% by weight.

Exposure to light, oxygen or heat can contribute to oxidation of VLCPUFAs, which can be prevented or minimized by methods known in the art. For example, storage of a tightly closed or sealed container or a pharmaceutical composition comprising a VLCPUFA in the dark or/and at reduced temperature (e.g., in a refrigerator) minimizes oxidation.

The therapeutically effective amount and frequency of administration of, and the length of treatment with, a compound of the disclosure (e.g., a VLCPUFA) to treat a medical condition described herein may depend on various factors, including the severity of the condition, the potency of the compound, the route of administration, the age, body weight, general health, gender and diet of the subject, and the response of the subject to the treatment, and can be determined by the treating physician.

In some embodiments, the effective daily dose of a compound of the disclosure (e.g., a VLCPUFA), whether in the form of an oral, parenteral (e.g., intravenous, subcutaneous or intramuscular) or topical (e.g., ophthalmic or transdermal) composition, is from about 0.1 to 100 milligrams (mg) per kilogram (kg) of body weight of the subject, which may be provided in a single dose or in divided doses (e.g., administered two, three or fours times a day to provide the total daily dose). In certain embodiments, the daily dose of a compound of the disclosure (e.g., a VLCPUFA) is from about 0.0001 mg/kg to 0.1 mg/kg (e.g., for diagnostic monitoring), from about 0.1 mg/kg to 2 mg/kg, or from about 2 mg/kg to 5 mg/kg. In other embodiments, the daily dose of a compound of the disclosure (e.g., a VLCPUFA) is from about 5 mg/kg to 10 mg/kg, from about 10 mg/kg to 20 mg/kg, from about 20 mg/kg to 30 mg/kg, from about 30 mg/kg to 40 mg/kg, from about 40 mg/kg to 50 mg/kg, from about 50 mg/kg to 75 mg/kg or from about 75 mg/kg to 100 mg/kg. In further embodiments, the effective daily dose of a compound of the disclosure (e.g., a VLCPUFA), whether in the form of an oral, parenteral (e.g., intravenous, subcutaneous or intramuscular) or topical (e.g., ophthalmic or transdermal) composition, is from about 10 mg to about 3 g, from about 50 mg to about 2 g, or from about 100 mg to about 2 g, or about 100-500 mg, about 500-1000 mg, about 1-1.5 g or about 1.5-2 g, which may be provided in a single dose or in divided doses (e.g., administered two, three or fours times a day to provide the total daily dose).

The frequency of administration of a compound of the disclosure (e.g., a VLCPUFA) can be, e.g., one or more times daily (e.g., 1, 2, 3, 4 or more times daily), once every two days or three times per week, once every three days or two times per week, weekly, bi-weekly or monthly. For example, an ophthalmic solution containing a compound of the disclosure (e.g., a VLCPUFA) can be administered by eye drop 1, 2, 3, 4 or more times daily, with each administration applying 1, 2, 3, 4 or more drops of the solution per eye. A reduced frequency of administration (e.g., weekly, bi-weekly or monthly) can be achieved by controlled- or sustained-release of the compound, such as via micelles or polymeric nanoparticles or via extended-release tablets or capsules.

Treatment with a compound of the disclosure (e.g., a VLCPUFA) can continue until, e.g., resolution of the condition being treated, such as substantial mitigation or elimination of one or more symptoms, complications or causes of the condition. In some embodiments, a symptom or complication of a condition is substantially mitigated if its severity, frequency or duration is reduced by at least about 30%, 50%, 75% or 90%. Therefore, the length of treatment with a compound of the disclosure (e.g., a VLCPUFA) can be, e.g., at least about 1, 2, 3, 4, 5 or 6 weeks, or at least about 1, 2, 3, 4, 5 or 6 months, or at least about 1, 2, 3, 4, 5 or 10 years.

A compound of the disclosure (e.g., a VLCPUFA) can be administered by any suitable route to treat a medical condition. Routes of administration include without limitation oral, parenteral (e.g., intravenous, subcutaneous, subdermal, intradermal, intramuscular, administration into the lumen or parenchyma of an organ, intraperitoneal, administration into a body cavity, intrauterine, and topical), topical (e.g., ocular, transdermal, buccal, sublingual, intranasal, pulmonary, anal/rectal and vaginal), and surgical administration. Topical administration of an active agent (e.g., a VLCPUFA) may deliver the active agent to the diseased tissue with minimal systemic distribution. A compound of the disclosure (e.g., a VLCPUFA) can be administered by any suitable means. Means of administration include without limitation tablets, capsules, powders, injections, implants, oral or nasal inhalation, transdermal delivery devices (e.g., patches), suppositories (e.g., rectal and vaginal suppositories), solutions, suspensions, emulsions, creams, gels, ointments, pastes, sprays, aerosols, particles, nanoparticles, microparticles, microspheres, and liposomes.

The disclosure further provides kits or packages containing a pharmaceutical composition comprising a compound of the disclosure (e.g., a VLCPUFA) and instructions for administering and using the compound to treat a medical condition. The composition can be, e.g., a solid oral dosage form such as a tablet or capsule; a sterile solution for parenteral (e.g., intravenous, subcutaneous or intramuscular) administration, which can be provided in, e.g., a pre-filled syringe; an ophthalmic solution provided in, e.g., an eye-drop bottle; or a composition provided in a transdermal patch.

Alternative to the carboxylic acid or salt form of a VLCFA (e.g., a VLCPUFA), the VLCFA (e.g., VLCPUFA) can be in the form of an ester, which can act as a prodrug. Esterases present in the blood and other body fluids, tissues and cells can quickly hydrolyze an ester of a VLCFA (e.g., a VLCPUFA) to the carboxylic acid or a salt thereof. Therefore, a pharmaceutical composition can contain an ester of a VLCFA (e.g., a VLCPUFA) and a pharmaceutically acceptable excipient or carrier, and an ester of a VLCFA (e.g., a VLCPUFA) can be used to treat any medical condition described herein. Accordingly, the entire disclosure of this application also applies to esters of VLCFAs (e.g., VLCPUFAs). Where an ester of a VLCFA (e.g., a VLCPUFA) has the general formula RC(═O)OR′, in some embodiments R′ is C1-C6 alkyl, such as methyl, ethyl, n-propyl, isopropyl, n-butyl, isobutyl, sec-butyl, tert-butyl, n-pentyl or n-hexyl.

### Therapeutic Uses

Age-related macular degeneration (AMD) affects the central vision, and with it, the ability to see fine details. In AMD, a part of the retina called the macula is damaged. Dry AMD is associated with the light-sensitive cells in the macula slowly breaking down. Wet AMD is associated with abnormal blood vessel growth beneath the retina. The blood vessels leak fluid and blood (hence the name wet AMD), which can create a large blind spot in the center of the visual field.

The ageing retina is prone to develop degenerative diseases, such as AMD. Changes in corneal toricity (curvature) cause alteration in refraction in the elderly, usually a change from the “with the rule” astigmatism to “against the rule” astigmatism. Other observations related to ageing include increased thickness of Descemet's membrane, cornea Farinata, white limbus girdle, mosaic degeneration, deep crocodile shagreen, Hassall-Henle bodies, arcus senilis, etc.

Hyperlipidemia is associated with high blood levels of lipids (or fats), such as cholesterol and triglycerides. One type of hyperlipidemia, hypercholesterolemia, is associated with high blood levels of non-HDL cholesterol and LDL (“bad”) cholesterol. Hypertriglyceridemia is a condition in which triglyceride blood levels are elevated. It is associated or exacerbated by uncontrolled diabetes mellitus, obesity and other conditions.

Hepatic steatosis is defined as intrahepatic fat of at least 5% of liver weight. Simple accumulation of triacylglycerols in the liver could be hepatoprotective. However, prolonged hepatic lipid storage may lead to liver metabolic dysfunction, inflammation, and advanced forms of non-alcoholic fatty liver disease (NAFLD). Non-alcoholic hepatic steatosis is associated with obesity, type 2 diabetes, and dyslipidemia.

Hyperglycemia, or high blood glucose, occurs when there is too much sugar in the blood. This can happen, for example, with low levels of insulin (the hormone that promotes absorption of glucose from the blood into liver, fat and skeletal muscle cells), or if insulin insensitivity/resistance occurs. Insulin resistance is when liver, fat and skeletal muscle cells do not respond well to insulin and cannot use glucose from the blood for energy.

Hyperglycemia is most often linked with diabetes. Broadly, hyperglycemia is associated with blood glucose greater than 125 mg/dL (milligrams per deciliter) while fasting (not eating for at least eight hours)—a person with a fasting blood glucose greater than 125 mg/dL has diabetes. In an aspect, a human subject for example has impaired glucose tolerance, or pre-diabetes, with a fasting blood glucose of 100 mg/dL to 125 mg/dL.

In an aspect, a human subject for example has hyperglycemia if their blood glucose is greater than 180 mg/dL one to two hours after eating. Postprandial means after eating a meal. Postprandial hyperglycemia is characterized by hyperglycemic spikes that induce oxidative stress.

Metabolic syndrome can be defined as a cluster of conditions that occur together, increasing the risk of heart disease, stroke and type 2 diabetes. These conditions include increased blood pressure, high blood sugar, excess body fat around the waist, and abnormal cholesterol or triglyceride levels.

Obesity is a complex disease condition involving an excessive amount of body fat. It is a medical condition that increases the risk of other diseases and health problems, such as heart disease, diabetes, high blood pressure, and certain cancers.

Subcutaneous adipose tissue diseases involving adipose tissue and its fascia, also known as adipofascial disorders, represent variations in the spectrum of obesity. The adipofascia diseases can be localized or generalized and include a common disorder primarily affecting women, lipedema, and four rare diseases, familial multiple lipomatosis, angiolipomatosis, Dercum disease, and multiple symmetric lipomatosis. The fat in adipofascial disorders is difficult to lose by standard weight loss approaches, including lifestyle (diet and exercise), pharmacologic therapy, and even bariatric surgery, due in part to tissue fibrosis.

Atherosclerosis is the thickening or hardening of the arteries caused by a buildup of plaque in the inner lining of an artery. Plaque deposits include fatty substances, cholesterol, cellular waste products, calcium, and fibrin. Plaque buildup causes the artery walls to become thickened and stiff. Atherosclerosis is a slow, progressive disease that may start as early as childhood but can also advance rapidly.

Cognitive health or function is the ability to clearly think, learn and remember, and is an important component of performing everyday activities. Cognitive impairment is associated with trouble with remembering, learning new things, concentrating, or making decisions that affect everyday life. Cognitive impairment ranges from mild to severe. With mild impairment, subjects may begin to notice changes in cognitive functions, but still be able to do their everyday activities. Severe levels of impairment can lead to losing the ability to understand the meaning or importance of something and the ability to talk or write, resulting in the inability to live independently.

Peroxisome proliferator-activated receptors (PPARs) are ligand-activated transcription factors of nuclear hormone receptor superfamily comprising the subtypes PPARα, PPARγ, and PPARβ/δ. They are involved in regulating glucose and lipid homeostasis, inflammation, proliferation and differentiation. VLCPUFAs can act as agonists, or otherwise activate the signaling pathway, of PPARs such as PPARα and PPARγ.

The disclosure provides a method of preventing or treating a medical condition responsive to treatment with a very long chain fatty acid (VLCFA, such as a VLCPUFA) in a patient, comprising administering a VLCFA (e.g., a VLCPUFA) or a salt thereof to the patient. Conditions responsive to treatment with a VLCFA (e.g., a non-deuterated or deuterated VLCPUFA) or a salt thereof include without limitation impaired retinal functions, macular degeneration (e.g., AMD), hyperlipidemia (e.g., hypercholesterolemia and hypertriglyceridemia), obesity, overweight, adipofascial disorders, hepatic steatosis, fatty liver disease (e.g., NAFLD), hyperglycemia (e.g., fasting hyperglycemia and postprandial hyperglycemia, impaired glucose tolerance, insulin resistance, pre-diabetes, diabetes (e.g., type 1 and type 2 diabetes), metabolic syndrome, cardiovascular diseases (e.g., coronary artery disease and atherosclerosis), hypertension, and impaired cognitive functions.

### EXAMPLES

### Animals and Experimental Design

All experiments were conducted in accordance with guidelines provided by the NIH Guide for the Care and Use of Laboratory Animals. Fish oil-derived very long chain polyunsaturated fatty acid (VLCPUFA) concentrate oil enriched in PUFA with carbon chain length longer than C22 was produced at Nippon Suisan Kaisha, Ltd. VLCPUFA oil concentrated in C24:5 n-3, C26:6 n-3, and C28:8 n-3 were produced from sardine oil. In a first step, cholesterol and environmental pollutant (e.g., dioxins) were removed from sardine oil by short-path distillation. In a further reaction step, ethyl ester was obtained in a transesterification with sodium ethoxide, and bleaching the fish oil was accomplished by treating the oil with bleaching clay (percent of process yield: 100%). The resulting fish oil ethyl ester was refined by molecular distillation, and fatty acids with carbon chain length less than 20 was removed (percent of process yield: 18.3˜19.7%). The ethyl ester was further purified by thin-film distillation to remove fatty acid ethyl esters with carbon chain length of 22, polymers, and diacylglycerol as much as possible (percent of process yield: 7%). The ethyl ester oil was further purified by high-performance liquid chromatography (HPLC) with methanol used for mobile phase, and ethyl esters of fatty acids with carbon chain length of 22 were further removed (percent of process yield: 0.6%). After the final silica gel purification process, peroxides were removed and VLCPUFA concentrate oil was obtained (percent of process yield: 94.4%). The levels of the major fatty acids in VLCPUFA concentrate oil are shown in Table 1.

The mouse experiments were approved by the Animal Care and Use Committee in the National Heart, Lung and Blood Institute, and at the University of Utah. Black C57BL/6J mice were obtained from Taconic Biosciences (Germantown, New York), and ApoE-KO mice were obtained from Jackson Lab (Bar Harbor, Maine). Animals were housed under a 12/12 light-dark cycle with access to food and water ad libitum. Twenty-four 7-month old C57BL/6J mice were fed on a chow diet AIN-93G (Harlan Teklad) supplemented with 1% (w/w) VLCPUFA oil, 5% (w/w) VLCPUFA oil, or not (control) for 8 weeks (n=8, 4 male and 4 female per group). In another study, twenty-four 9-month old ApoE-KO mice were fed on a chow diet AIN-93G (Harlan Teklad) supplemented with 1% (w/w) VLCPUFA oil, 3% (w/w) VLCPUFA oil, or not (control) for 8 weeks (n=8, 4 male and 4 female per group), and the age-matched C57BL/6J mice were used as the wild-type control. Body weight was monitored every 2-3 weeks in 5 hr-fasting animals. After 8 weeks of food consumption, the mice were euthanized via intraperitoneal injection of tribromoethanol (avertin). Blood samples were collected via the retro-orbital plexus, and plasma was separated by centrifugation. Tissue samples were snap-frozen and stored at −80° C. until further use. An additional aliquot of liver, brain, kidney, spleen, heart, skeletal muscle, small intestine, and testis (male) was fixed in 10% formalin. In single-dose gavage experiments, twenty-five 3-month-old male mice were oral gavaged with 100 μL of VLCPUFA oil that was prepared with a liposome kit (Sigma-Aldrich). Alpha-tocopherol (0.025%) was added to prevent oxidation of VLCPUFA. A dose of 6 mg/day/mouse as VLCPUFA (250 mg/kg body weight) was used, comparable with the dosage for the purified C32:6 n-3 in a previous study [Gorusupudi et al., Proc. Natl. Acad. Sci. USA, 118(6):e2017739118 (2021)]. The animals were euthanized at time points of 0, 2, 4, 8 and 24 hr (n=5 at each time point), and the retina was separated from eyes dissected under a microscope. Lipid was extracted from one pair of retina/mouse for further GC-MS analysis as previously described [Liu et al., J. Lipid Res., 51(11):3217-3229 (2010)]. In repeated-dose gavage experiments, 3-month-old male mice were randomly divided into the two following treatment groups: VLCPUFA concentrate oil (n=6) and vehicle (control) (n=6). Liposome (control) and VLCPUFA oil (80 mg/[kg·d]) were administered to the mice by oral gavage once daily. After 15 days of the experimental period, 6 hr-fasted mice were sacrificed for harvesting eye tissues, and retina and retinal pigment epithelium (RPE) were separated for GC-MS analysis.

Half-maximal effective concentration (EC50) refers to the concentration of a drug, antibody or toxicant which induces a response halfway between the baseline and maximum after a specified exposure time.

### Electroretinograms and Visual Behavior Testing

Electroretinograms (ERGs) and visual behavior exam were used to assess the retinal function of mice after 15 consecutive days of oral gavage of VLCPUFA oil as described previously [Gorusupudi et al. Proc. Natl. Acad. Sci. USA, 118(6):e2017739118 (2021)]. Briefly, mice were dark-adapted overnight prior to ERG recording, and procedures were conducted in a light-proof room with the aid of a dim red light. Upon induction of anesthesia and mydriasis, mice were placed on a thermostatically controlled heating pad, and a gold ERG electrode was placed upon the central cornea. Stimulus response functions were obtained under dark- and then light-adapted conditions, and ERG a-wave and b-wave amplitudes in multiple flash luminances were measured and analyzed. In addition, at the end of repeated gavage of VLCPUFA oil or vehicle for 15 days, the optokinetic behavioral tests were performed to analyze the visual function of mice. Visual acuity was measured using an optokinetic testing system (OptoMotry from Cerebral Mechanics), a validated non-invasive methodology. In brief, the tracking response (optokinetic reflex) was recorded to a rotating visual stimulus displayed on LCD panels surrounding the mouse, and visual acuity was measured at 100% contrast.

### Biochemical Assays

Plasma triglyceride (TG), cholesterol, glucose, bilirubin, chloride, calcium, magnesium, creatine, alkaline phosphatase, aspartate aminotransferase (AST), alanine aminotransferase (ALT), potassium, albumin, total creatine kinase, lactate dihydrogen, total protein, amylase, inorganic phosphorus, urea nitrogen, and uric acid were measured with use of an ILab 600 automatic analyzer and enzyme-based kits and quality controls. Plasma insulin (R&D Systems, Minneapolis, Minnesota) and adiponectin (R&D Systems) were determined with enzyme-linked immunosorbent assay (ELISA) kits. Hepatic total lipids were extracted with the mixture of chloroform and methanol (2:1) as described previously [Folch et al., J. Biol. Chem., 226:497-509 (1957)], and the content of TG and total cholesterol were measured by colorimetric assays (Wako Chemicals, Richmond, Virginia).

### Fast Protein Liquid Chromatography (FPLC) Profile Analysis

Pooled plasma (150 μL) from each diet group (n=8) was injected onto Superose FPLC column (GE Healthcare). Lipoproteins were eluted with elution buffer and 0.5 mL fractions were collected at a flow rate of 0.5 mL/min. Total cholesterol and phospholipids levels were measured in FPLC samples according to manufacturer instructions (Wako Chemicals).

### Histological Analysis

After formalin fixation, liver, brain, kidney, spleen, heart, skeletal muscle, small intestine, and testis (male) were embedded in paraffin and sectioned at 5 m. All slices were stained with hematoxylin and eosin (H & E) for microscopic examination. Additionally, Oil-red O stain was performed on fresh frozen sections (10 μm) to illustrate hepatic lipid accumulation. All slides were then scanned with Hamamatsu NDP scanner for histology evaluations by a pathologist in a blinded manner.

### Lipidomic Analysis

Characterization of the incorporation of VLCPUFA into specific tissues was performed by untargeted lipidomic and metabolomic approaches, using HRAM-MS and MS/MS. To determine utilization of VLCPUFA by incorporation into complex lipids, global untargeted lipidomic analysis was used to identify and quantify individual molecular species of complex lipids including mono-, di-, and triacylglycerols; the phospholipid classes PA, PC, PE, PG, PI, PS, and their lyso and ether-linked subclasses; free fatty acids; free and esterified cholesterol; and the major sphingolipids sphingomyelin, ceramide, hexosyl ceramide, and lactosyl ceramide, as previously described [Busik et al. Methods Mol. Biol., 579:33-70 (2009)]. All LC-MS features were subjected to statistical analysis by principal component analysis (PCA) and partial least squares-discriminant analysis (PLS-DA) approaches.

### Statistical Analysis

All statistical analyses were performed using GraphPad Prism statistical software (GraphPad Software, version 6). A one-way ANOVA with Tukey's post hoc test for multiple comparisons was used to compare the difference among multiple groups, and a two-tailed Student's t-test was used for the comparison between two groups. Results are presented as mean±SE and the significant level was set at 0.05.

### Safety of Dietary VLCPUFA

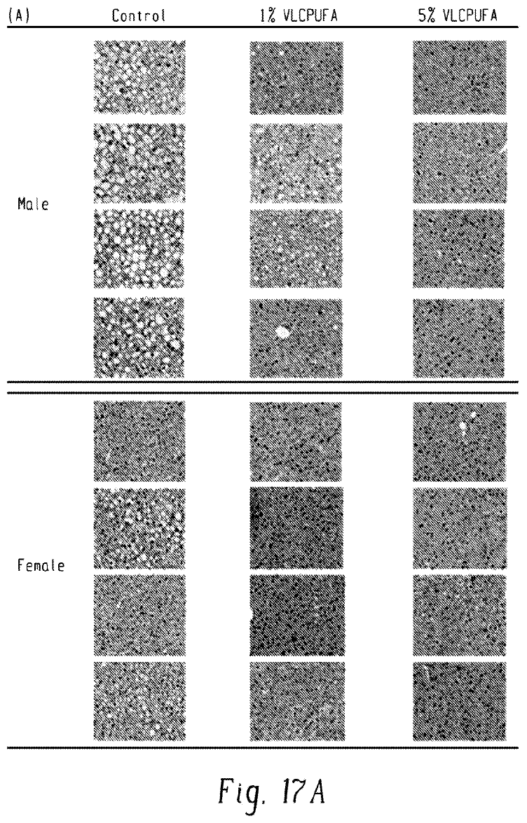

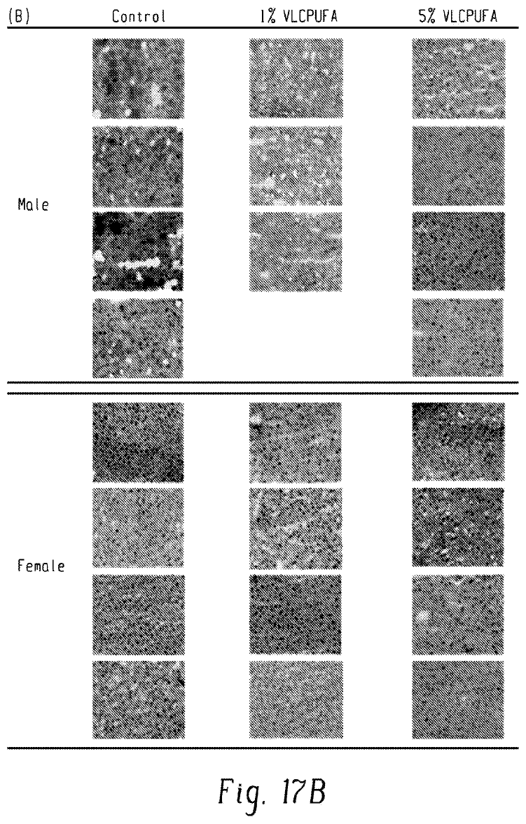

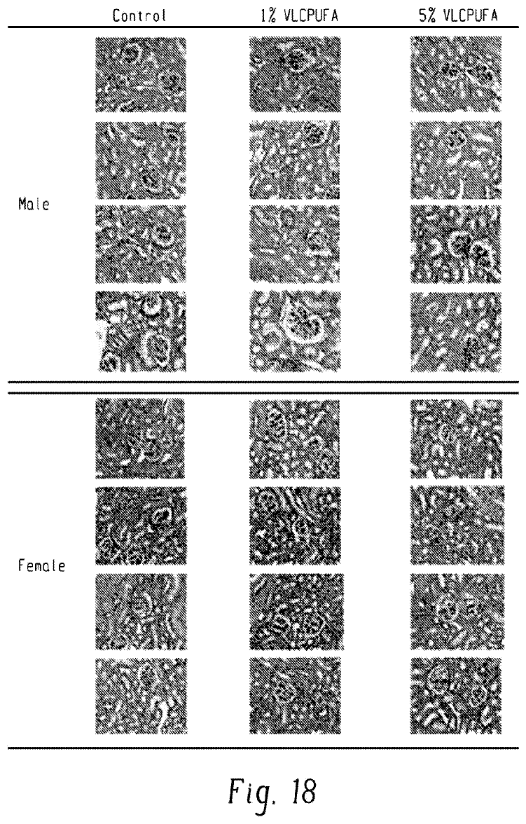

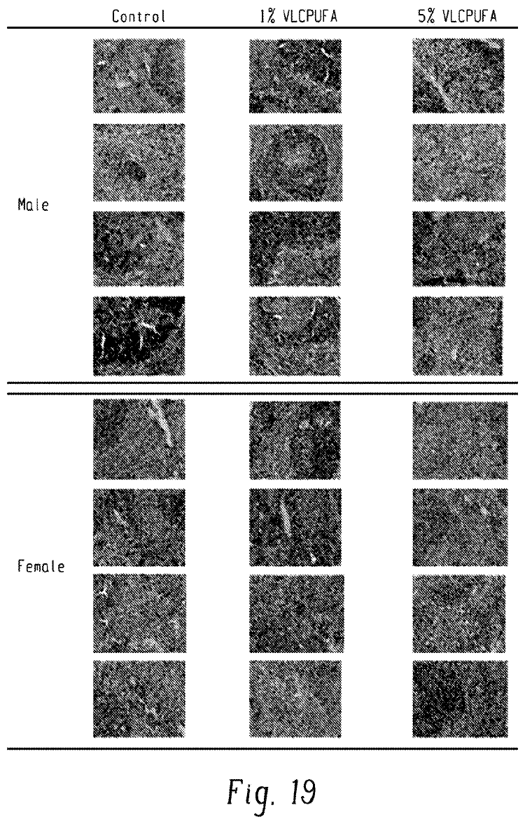

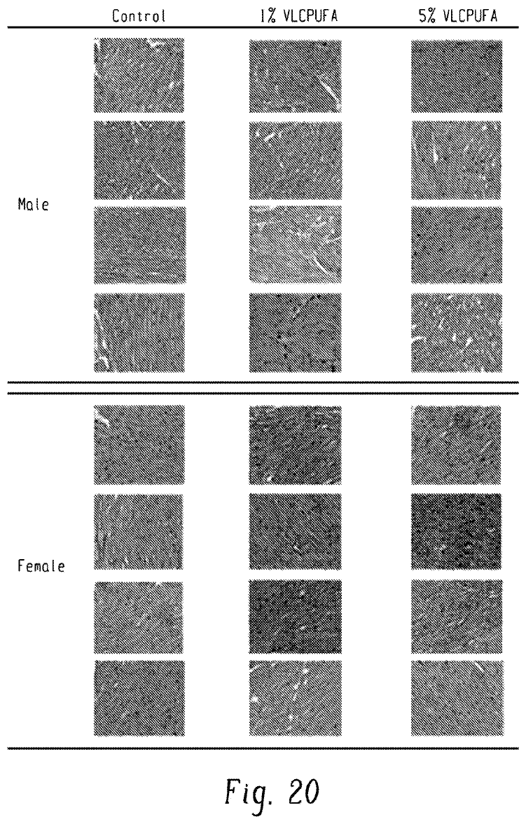

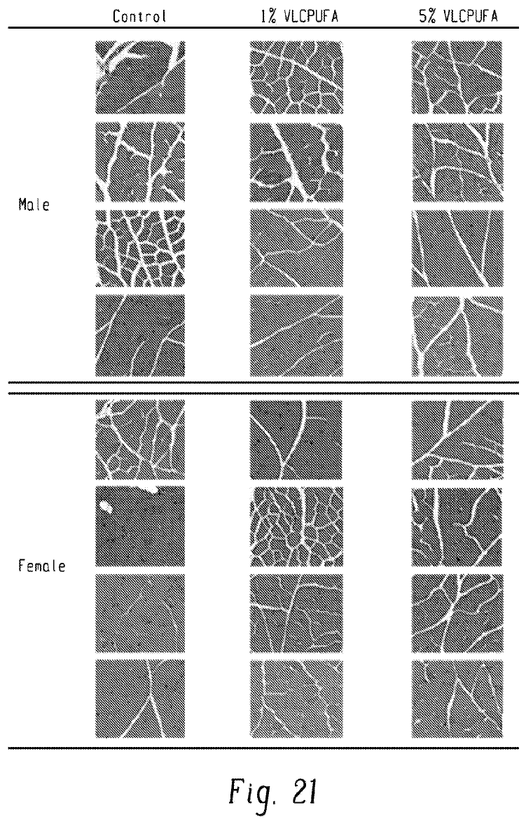

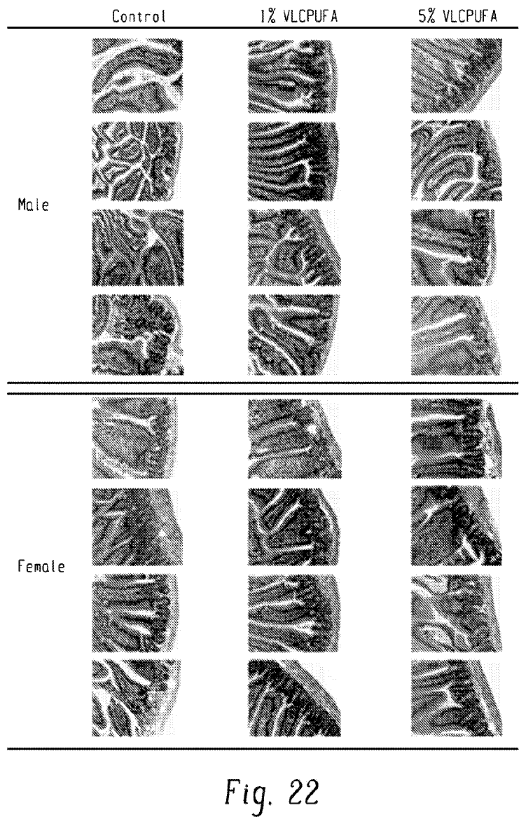

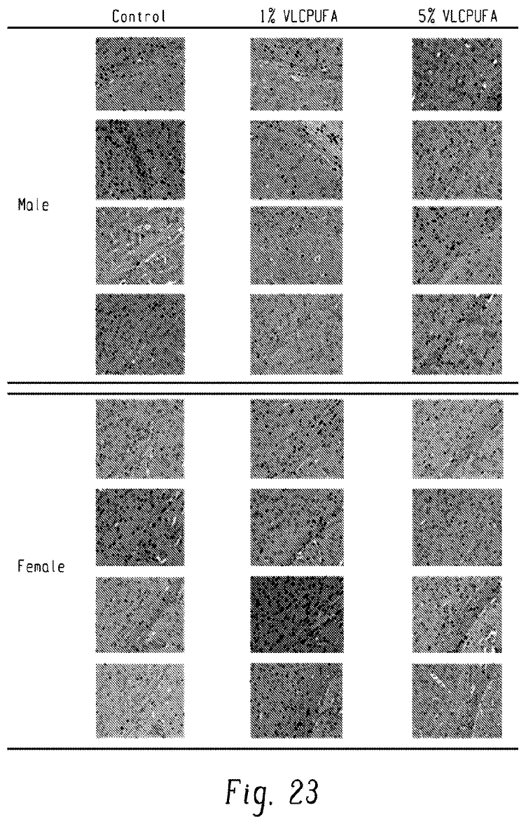

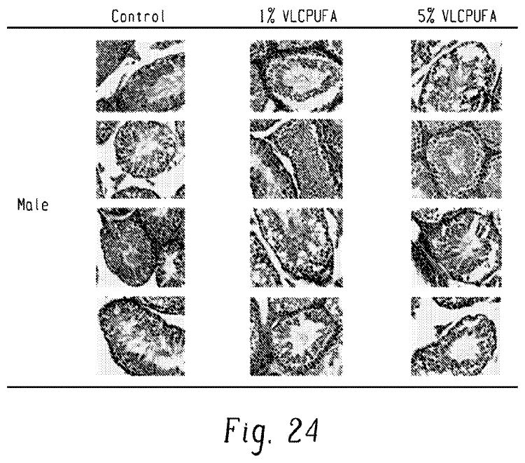

General appearance of the mice was monitored throughout the experimental period in all studies, and no abnormalities were noted. Body weight measurements were recorded every two weeks in a 2-month free-feeding study, and no significant changes were found over time in any of the diet groups. There were significant differences in plasma metabolic panel test results between control and VLCPUFA diet groups starting with alkaline phosphatase levels, except for lactate dihydrogen levels (Table 2). In addition, H & E-stained sections of liver, kidney, spleen, heart, skeletal muscle, small intestine, brain and testis were examined at the end of the 2-month feeding study, and no abnormalities were found in either VLCPUFA-treated or control mice (FIG. 17-24).

### Fatty Acid Composition in Retina and RPE

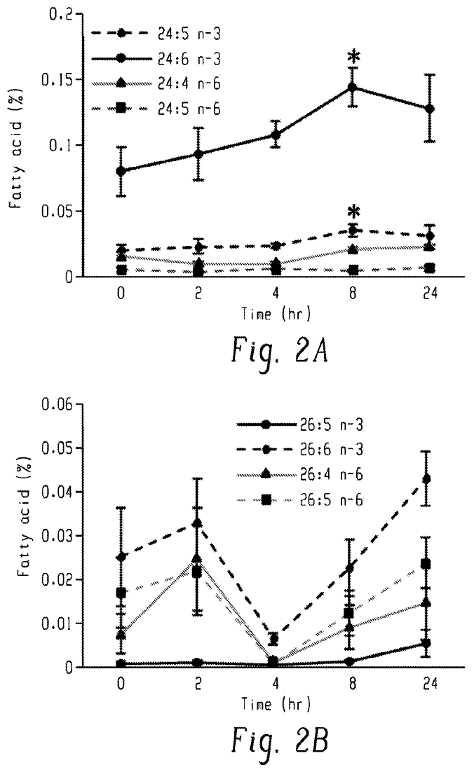

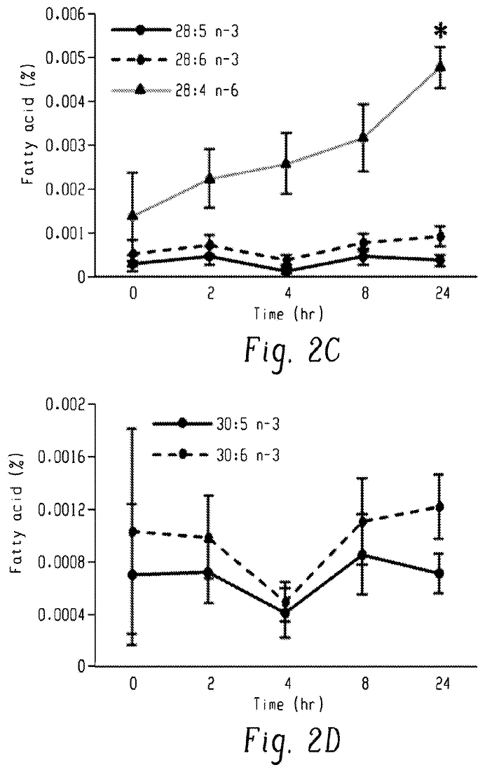

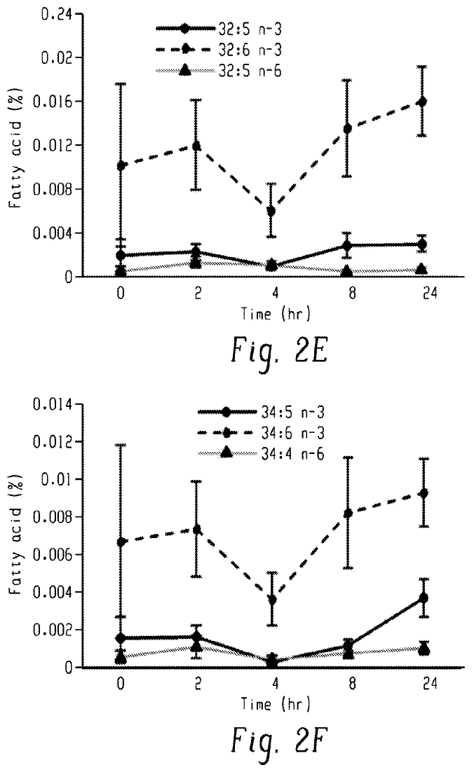

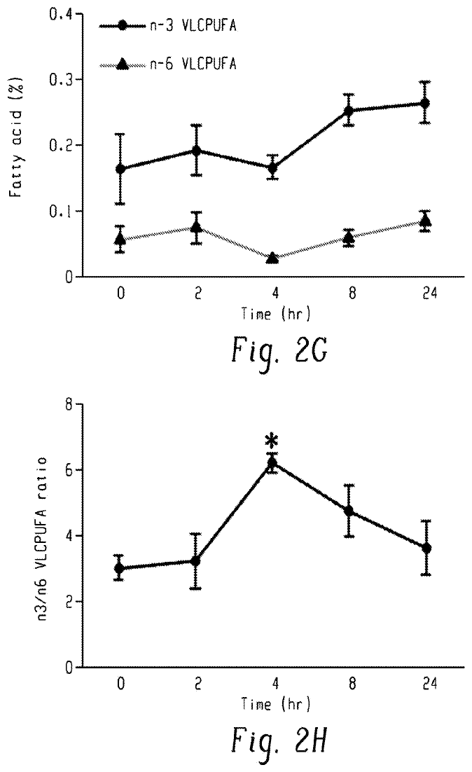

In single-dose gavage study, the retina level of C24:5 n-3 rose by 70% (P<0.05) and that of C24:6 n-3 increased by 79% (P<0.05) at 8 hr after oral administration of VLCPUFA oil, and then gradually declined to essentially baseline levels at 24 hr (FIG. 2A). There was no significant difference in retina levels of n-3 C26-C34 VLCPUFAs at any time point (FIG. 2B-2F). Retina levels of n-6 C24-C34 VLCPUFAs remained essentially unchanged throughout all the time periods (FIG. 2A-2F), except that C28:4 n-6 increased from 0.0014% at baseline to 0.0048% at 24 hr after VLCPUFA treatment. There was a tendency towards decreased total n-6 VLCPUFA retina levels at 4 hr compared with baseline, resulting in a 2-fold increase in n-3/n-6 VLCPUFA ratio from 0 hr to 4 hr after VLCPUFA treatment.

Table 3 shows the retina and retinal pigment epithelium (RPE) levels of shorter-chain (C<24) fatty acids after 2 weeks of repeated gavage of VLCPUFA oil in C57BL/6J mice. There were no significant differences in the retina and RPE levels of total saturated fatty acids and mono-unsaturated fatty acids (MUFAs) between the control and VLCPUFA groups except for the retina levels of saturated fatty acids, although C14:0, C16:0 and C16:1 levels increased significantly in the retina in the VLCPUFA group. Repeated gavage of VLCPUFA oil for 2 weeks significantly decreased total n-6 PUFAs levels by 11% and 25% in the retina and RPE, respectively (P<0.05). Total n-3 PUFAs levels were also significantly reduced in the retina, but not in the RPE, compared with control. Thus, the significant decrease in RPE n-6 PUFAs level coupled with the lack of significant change in RPE n-3 PUFAs level resulted in the notable increase in RPE n-3/n-6 PUFAs ratio compared with control (P<0.05).

After 2 weeks of repeated gavage of VLCPUFA oil in C57BL/6J mice, Table 4 shows that there were not notable differences in retina and RPE levels of individual and total n-3 and n-6 C24-C34 VLCPUFAs between the control and VLCPUFA groups, except for significant increases in RPE level of 26:5 n-6 and in retina and RPE levels of C28:4 n-6 with VLCPUFA oil treatment, and significant reductions in retina and RPE levels of C26:6 n-3 and in RPE level of 34:6 n-3 with VLCPUFA oil treatment. Repeated gavage of VLCPUFA oil for 2 weeks significantly reduced the levels of total n-6 C18-C34 PUFAs by 12% (P<0.05) and 24% (P<0.0001) in the retina and RPE, respectively, significantly reduced retina levels of total n-3 C18-C34 PUFAs by 11% (P<0.05), and significantly increased the n-3/n-6 C18-C34 PUFAs ratio by 38% in the RPE (P<0.001).

### Effect of Dietary VLCPUFA on Visual Function

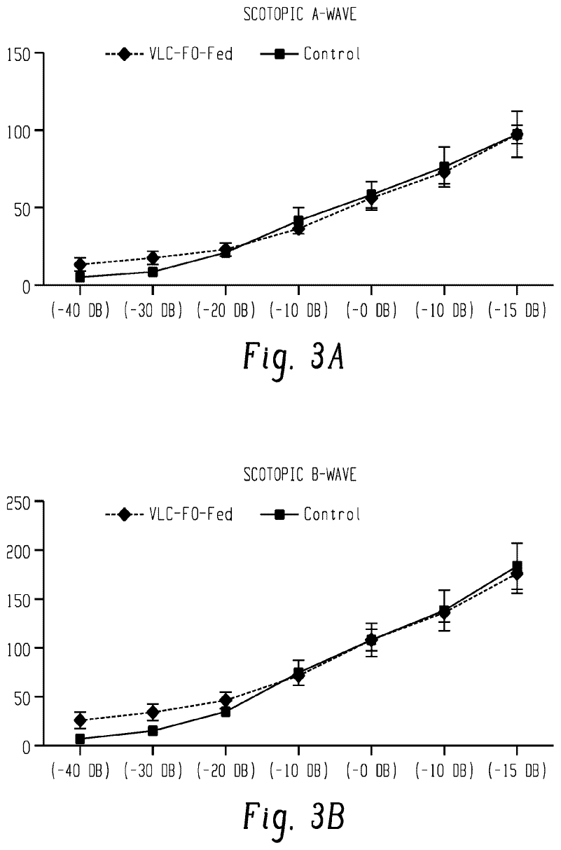

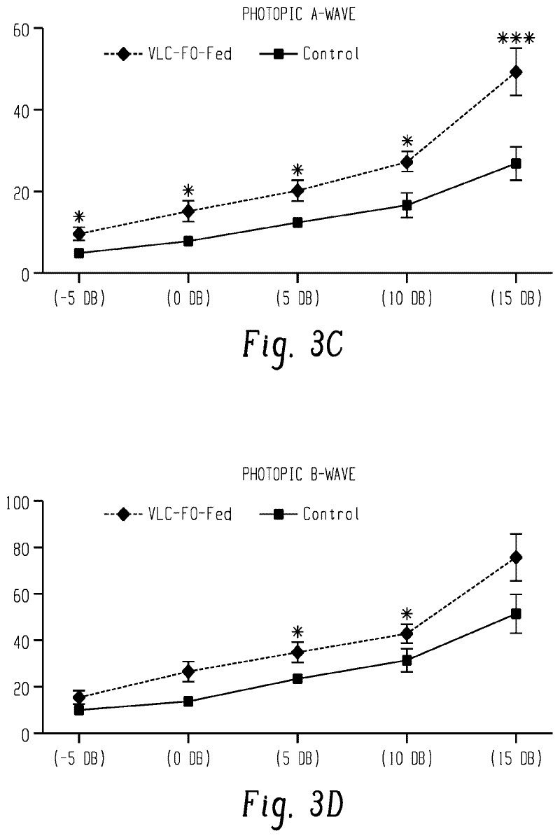

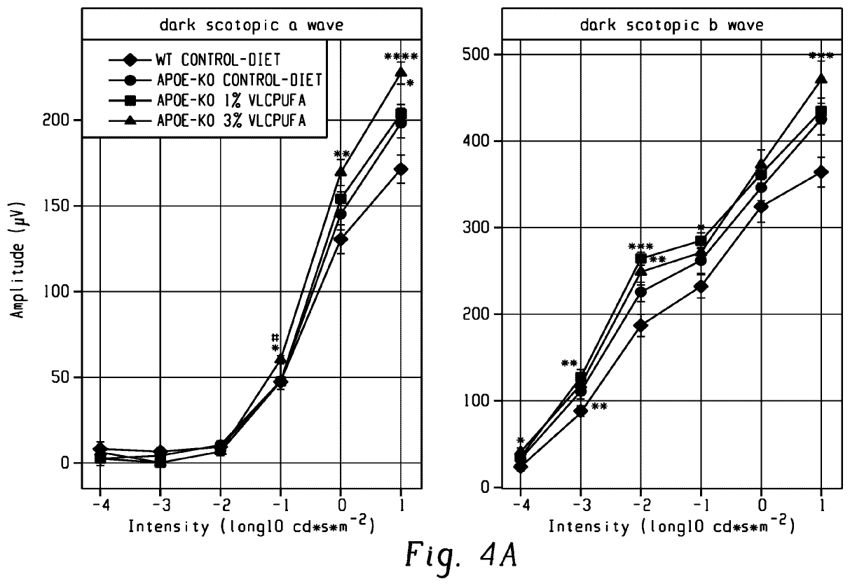

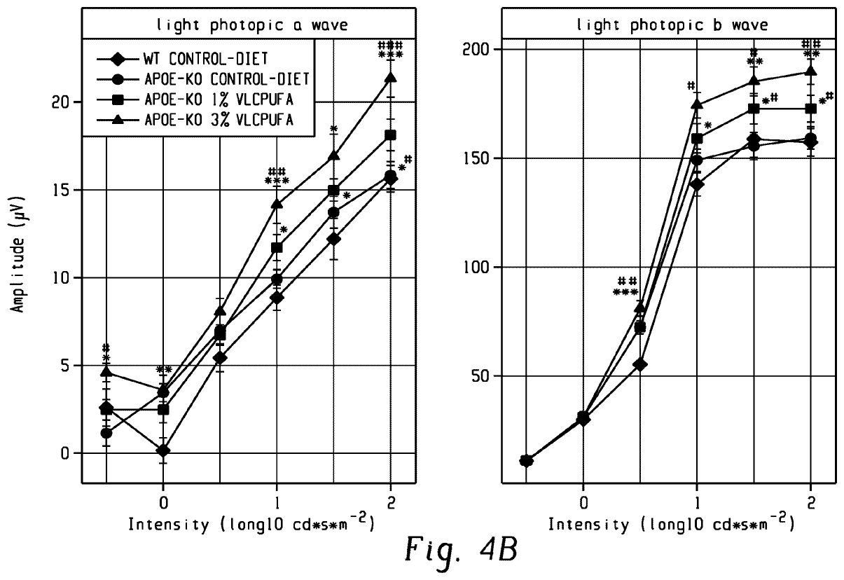

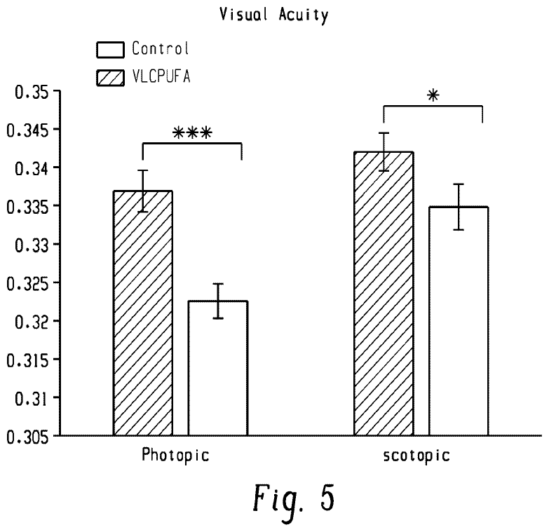

The ERG records showed that repeated gavage of VLCPUFA for 2 weeks resulted in a 78% and 52% increase on average in the amplitude of the photopic a-wave and b-wave, respectively (FIGS. 3C and D). In another 8-week intervention study, dietary VLCPUFA dose-dependently improved photopic and scopotic a-wave and b-wave (FIGS. 4A and B). In addition, VLCPUFA-treated mice exhibited a small but significant increase in photopic (P<0.001) and scotopic (P<0.05) visual acuity compared with control (FIG. 5).

### Effect of Dietary VLCPUFA on Plasma Lipids, Lipoproteins, Glucose and Insulin

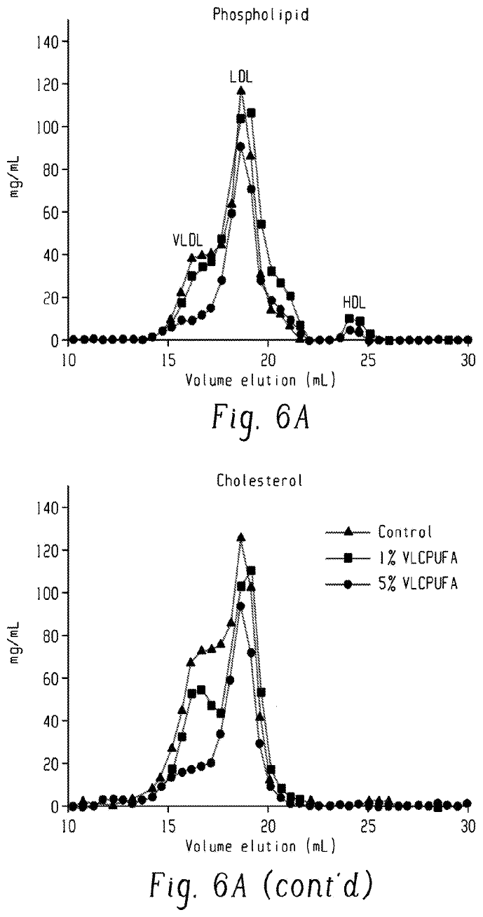

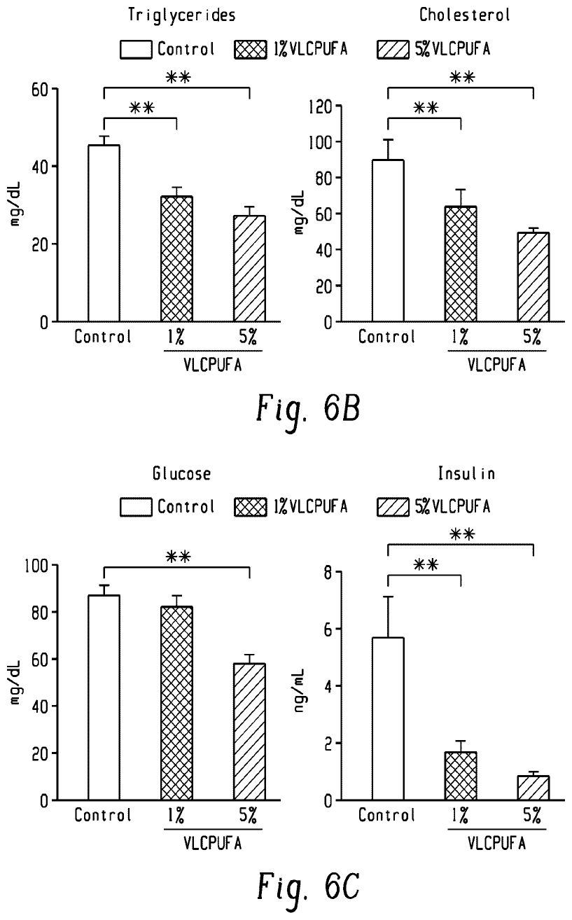

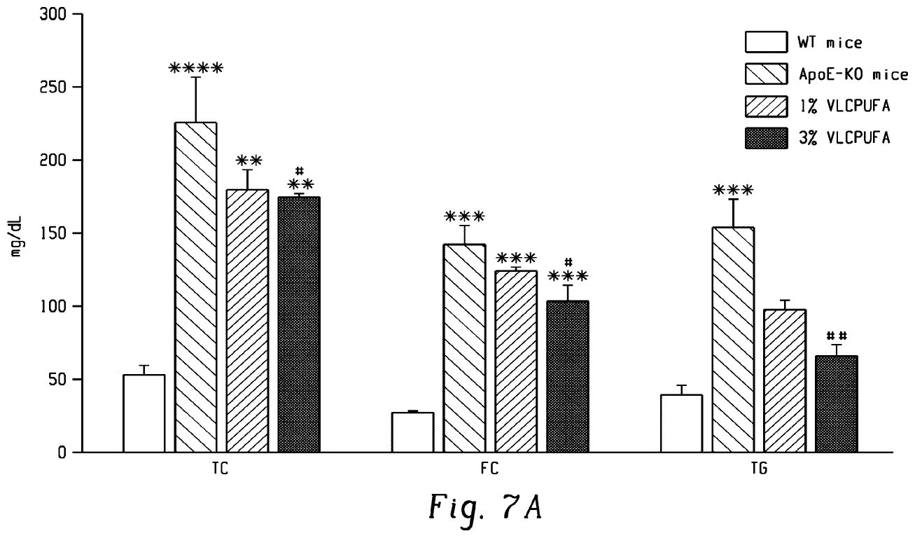

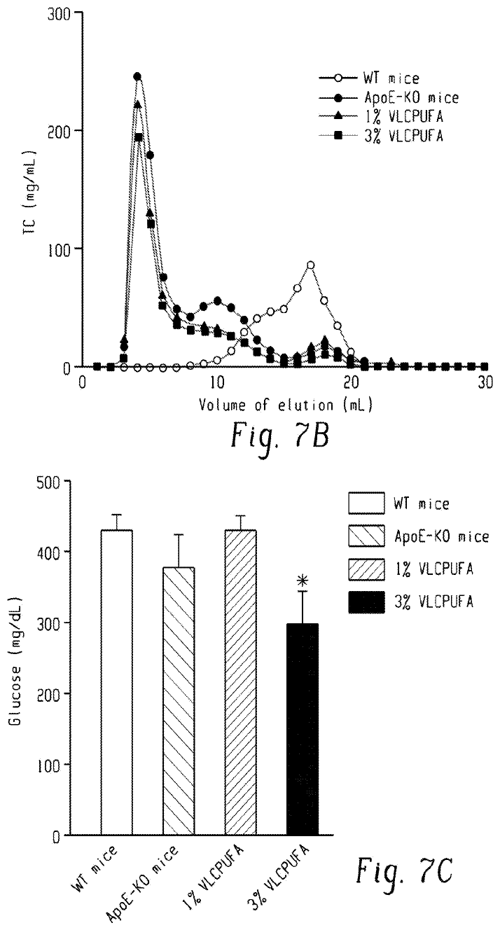

After the 8-week intervention period, dietary VLCPUFA dose-dependently lowered plasma triglycerides and total cholesterol levels compared with the control diet in C57BL/6J mice (P<0.05) (FIG. 6B). Analysis of the lipoprotein profiles revealed that the decreases were due to decreases in VLDL and LDL cholesterol and phospholipid levels compared with the control (FIG. 6A). Plasma levels of glucose and insulin were significantly decreased in the 5% VLCPUFA group as compared with the control group or the 1% VLCPUFA group (P<0.05) (FIG. 6C). In ApoE-KO mice, VLCPUFA supplementation also decreased lipid contents in VLDL and LDL (FIG. 7B), and dose-dependently decreased plasma cholesterol and triglyceride levels (FIG. 7A).

### Effect of Dietary VLCPUFA on Hepatic Lipid Accumulation

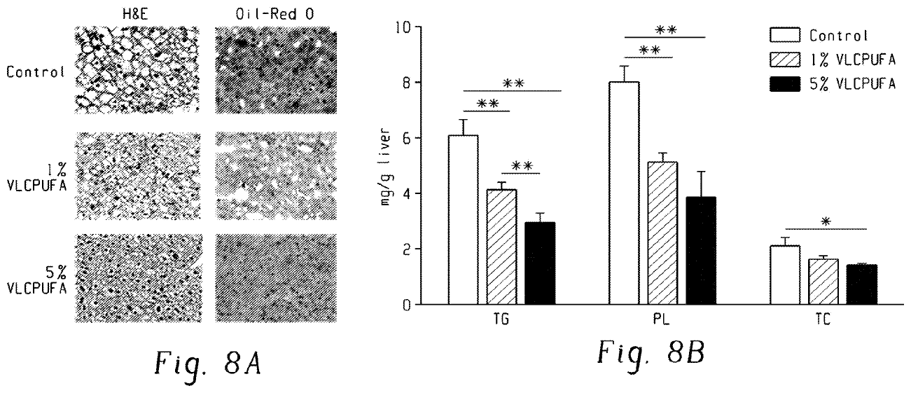

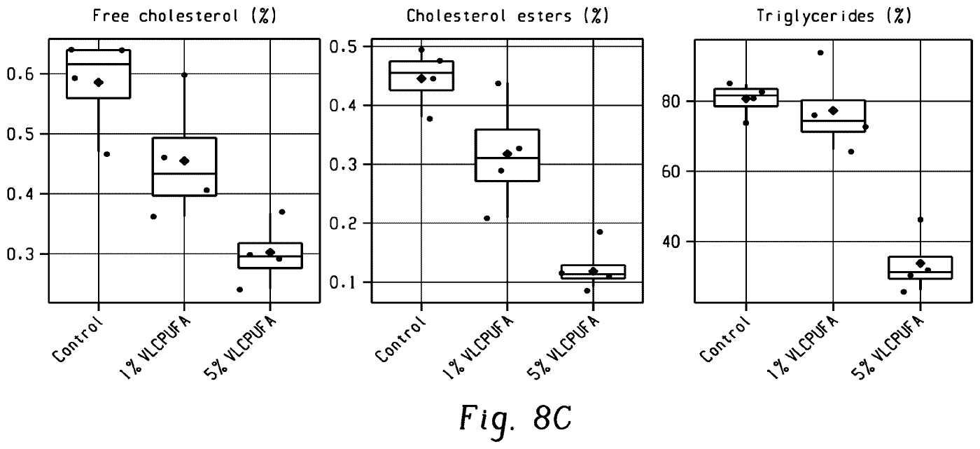

A decreased level of Oil-Red-O staining with smaller and fewer lipid droplets in liver tissues was detected in the 1% and 5% VLCPUFA groups, with a greater decrease in the 5% VLCPUFA group (FIG. 8A). The data of H & E staining validated the results of Oil-Red-O staining (FIG. 8A). The quantification of liver lipid content showed a dose-dependent decrease in hepatic triglycerides (TG), phospholipid (PL) and cholesterol (TC), as measured enzymatically (FIG. 8B) or by LC-MS/MS (FIG. 8C).

### Effect of Dietary VLCPUFA on Tissue-Specific Lipidomic Profile

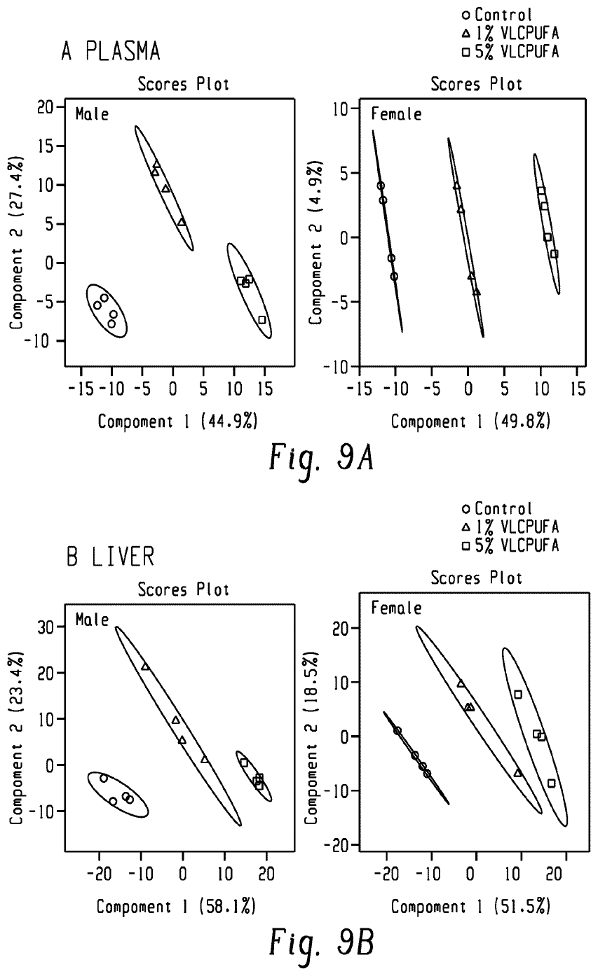

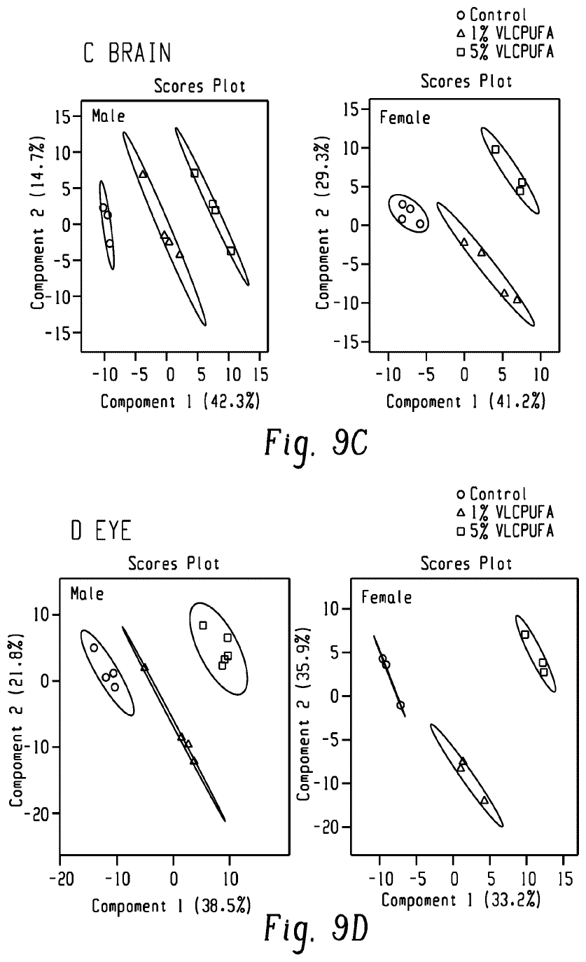

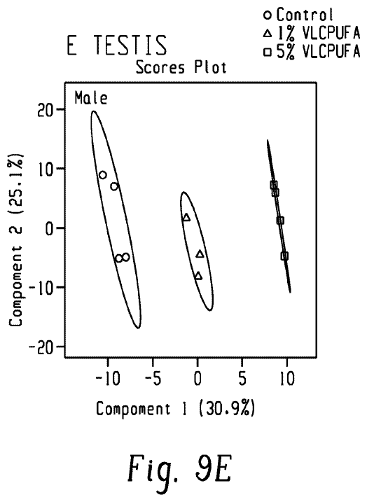

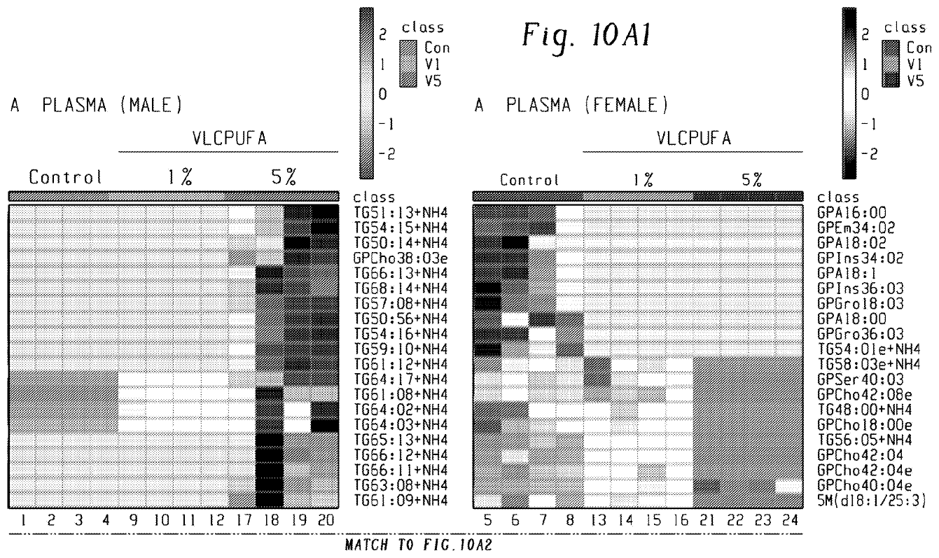

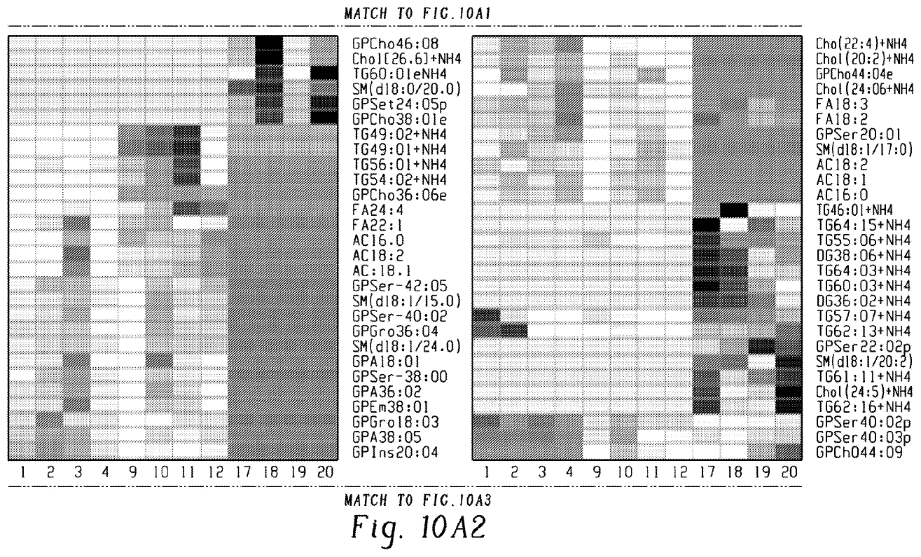

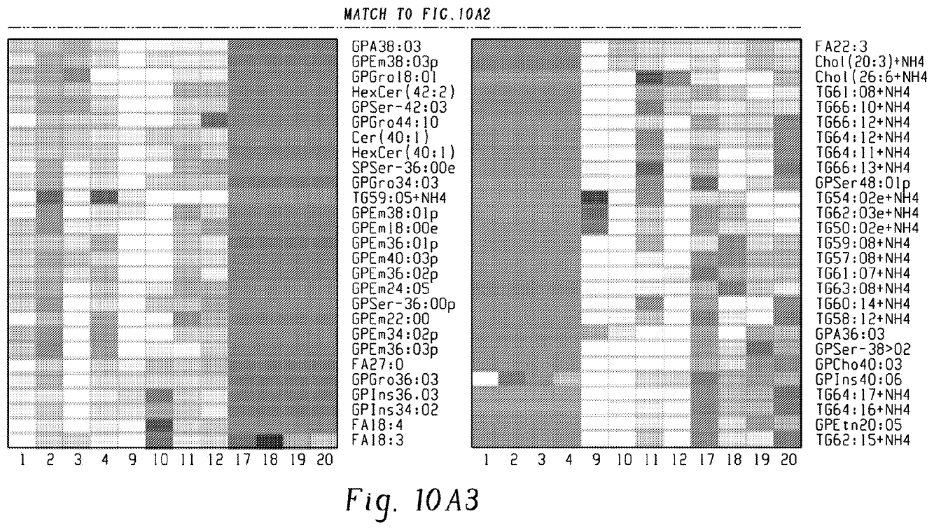

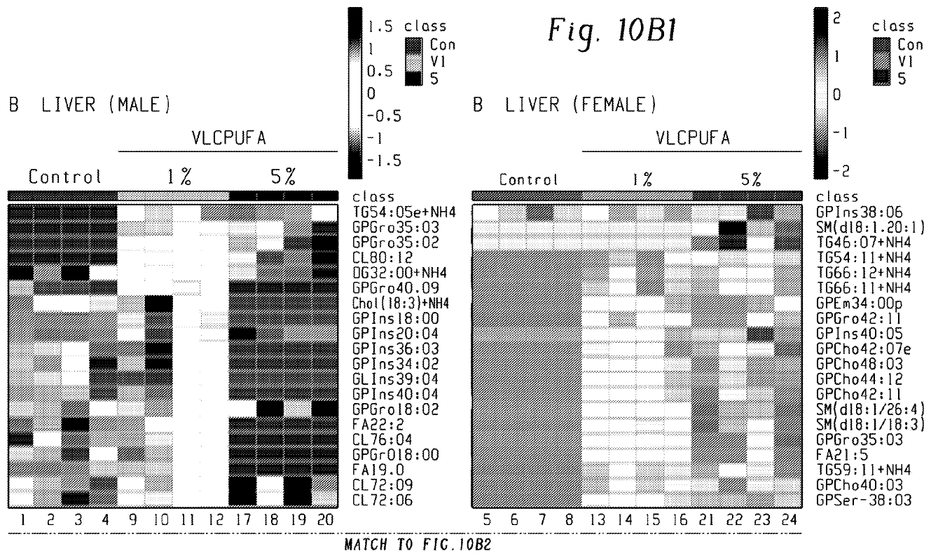

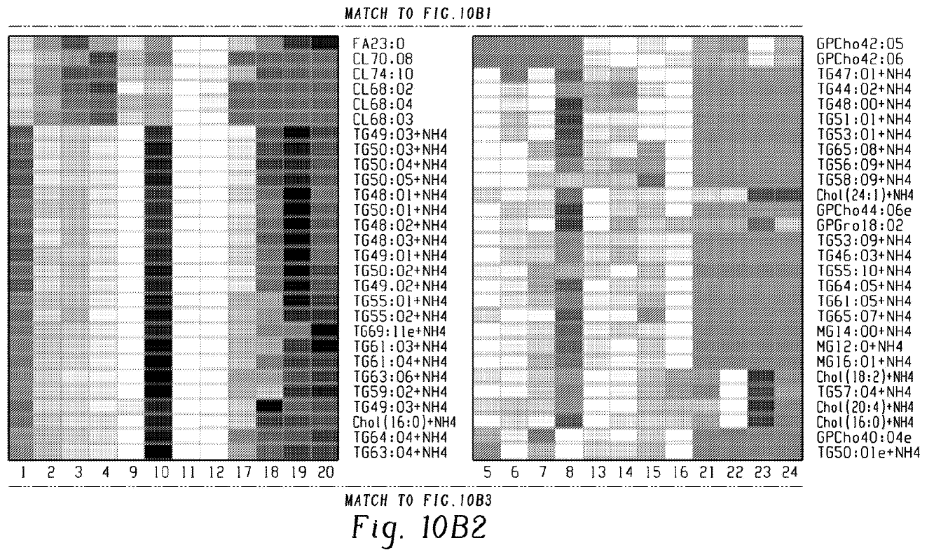

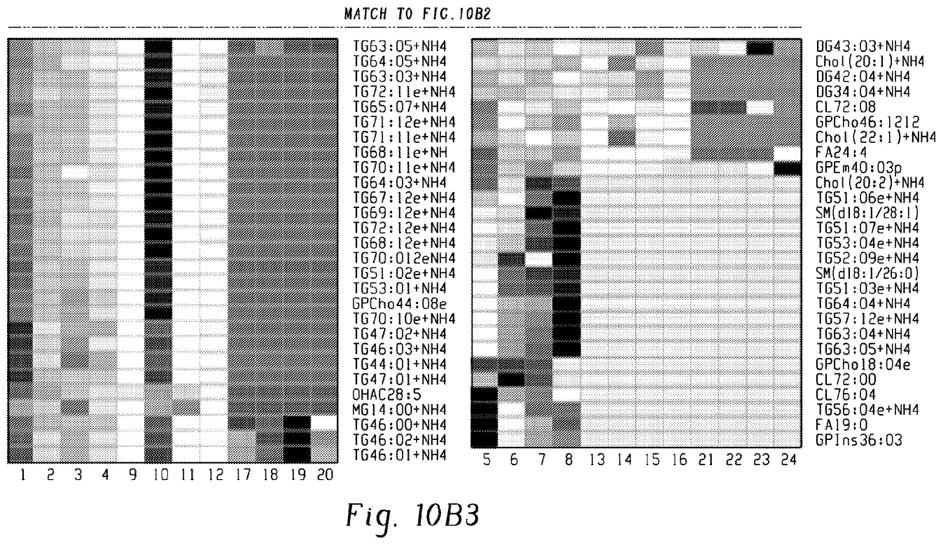

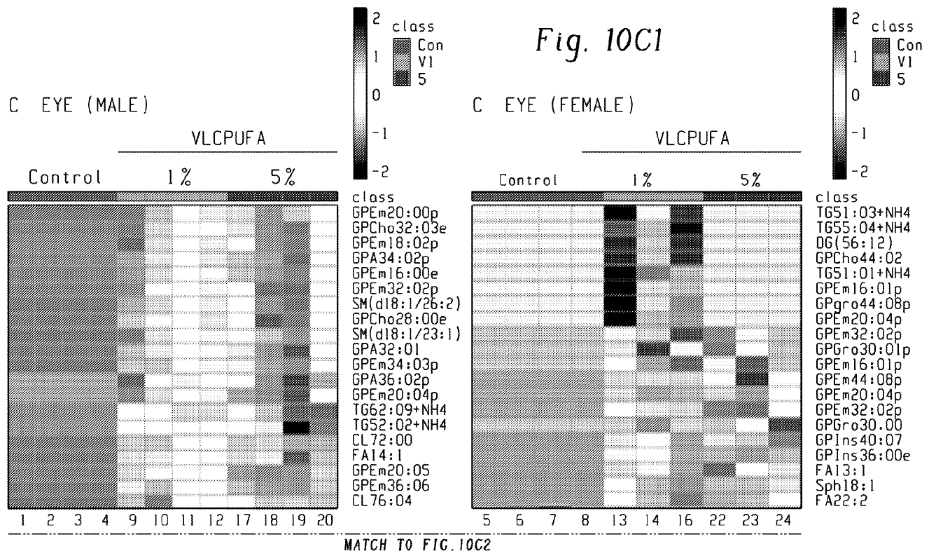

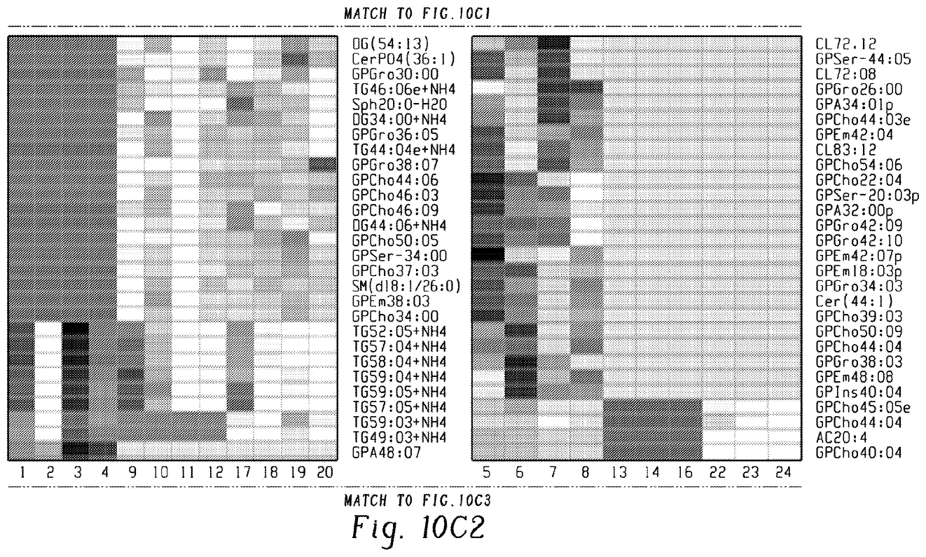

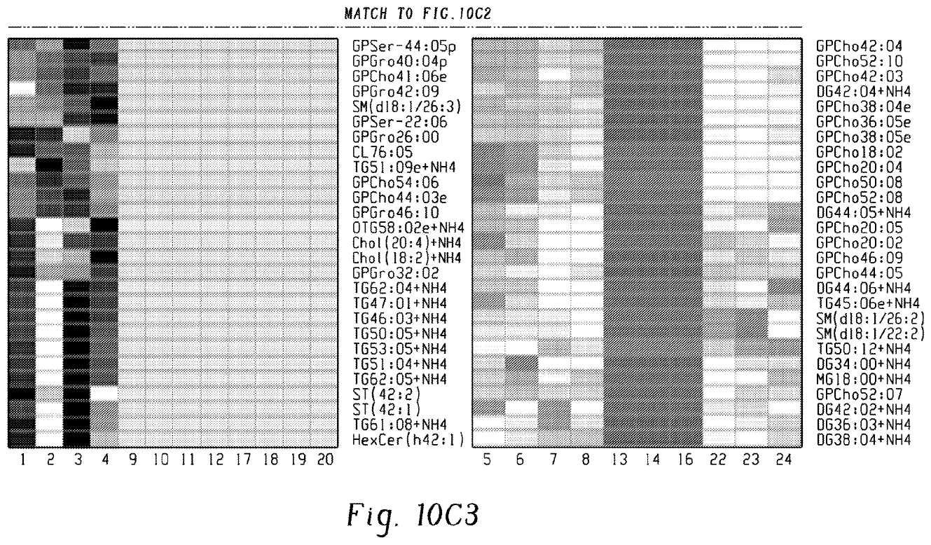

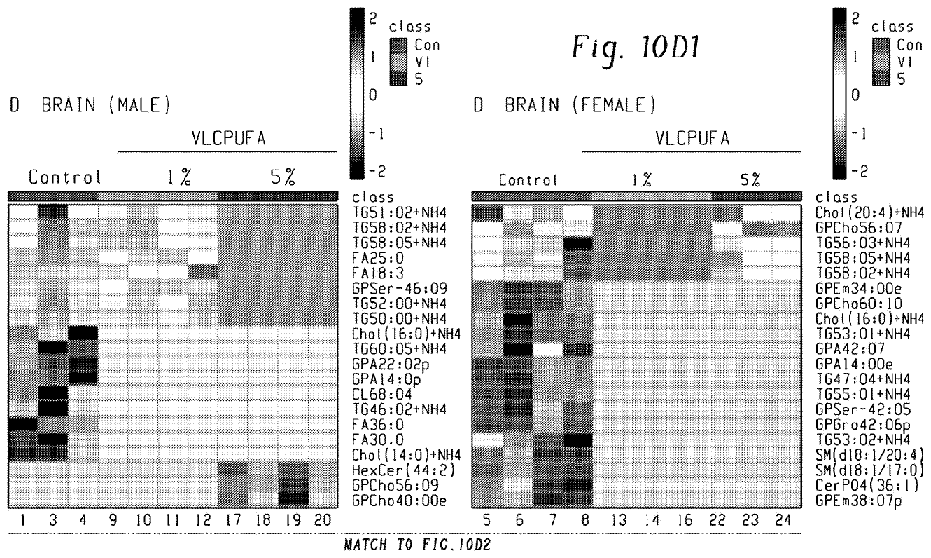

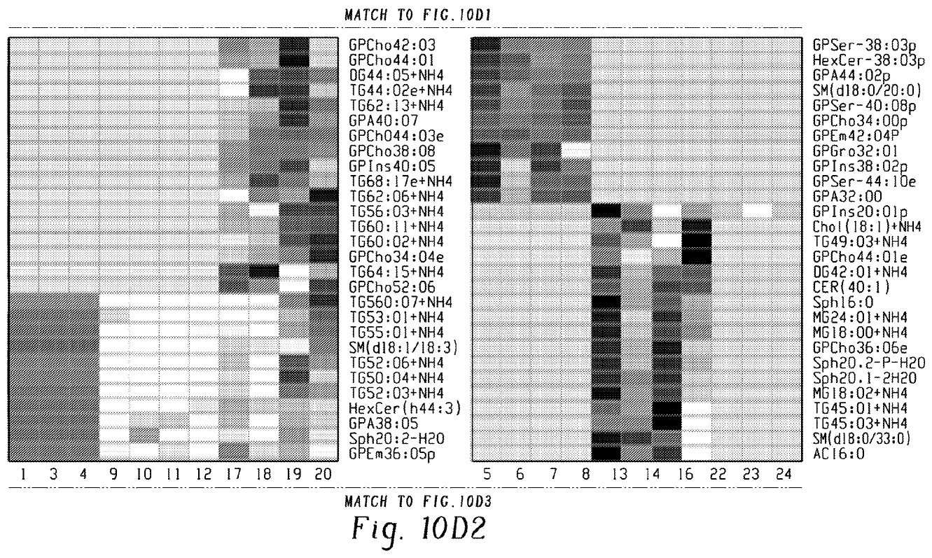

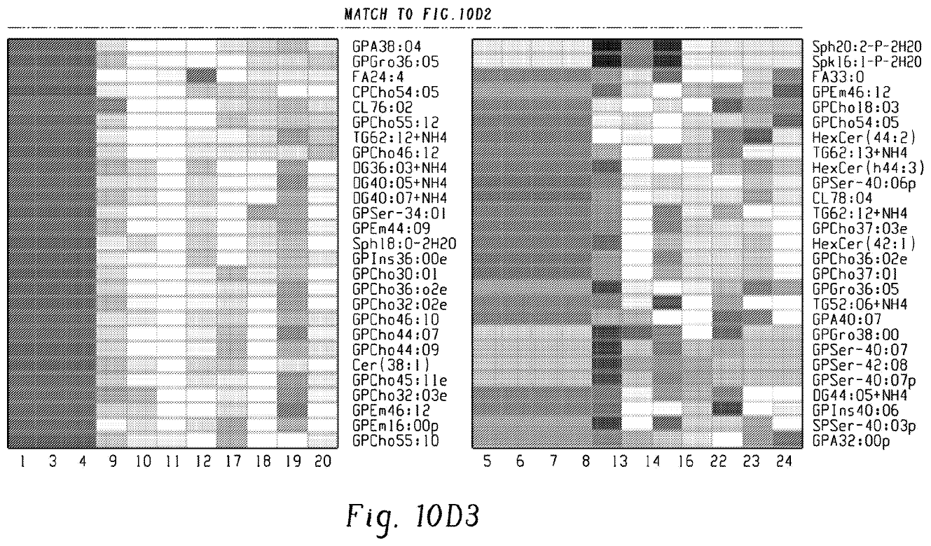

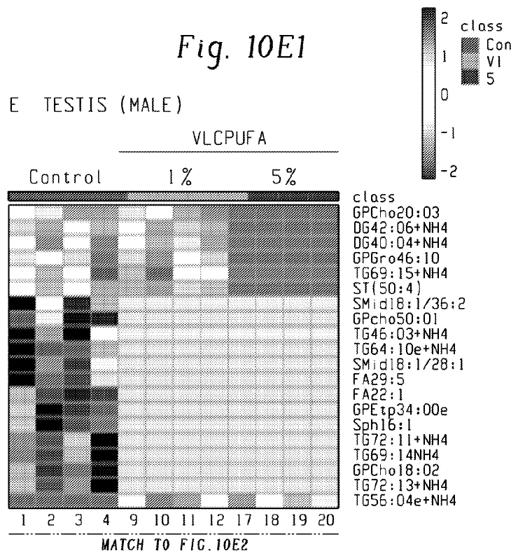

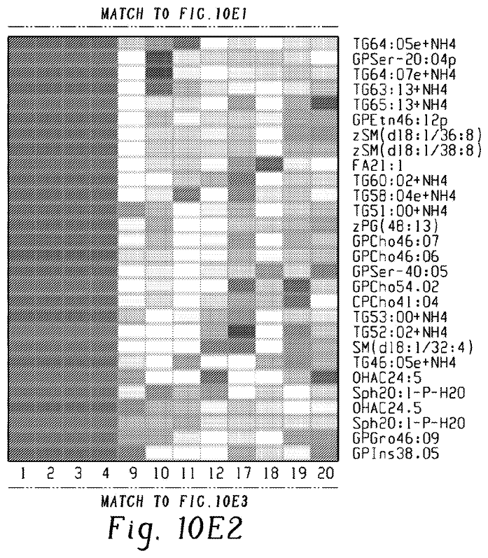

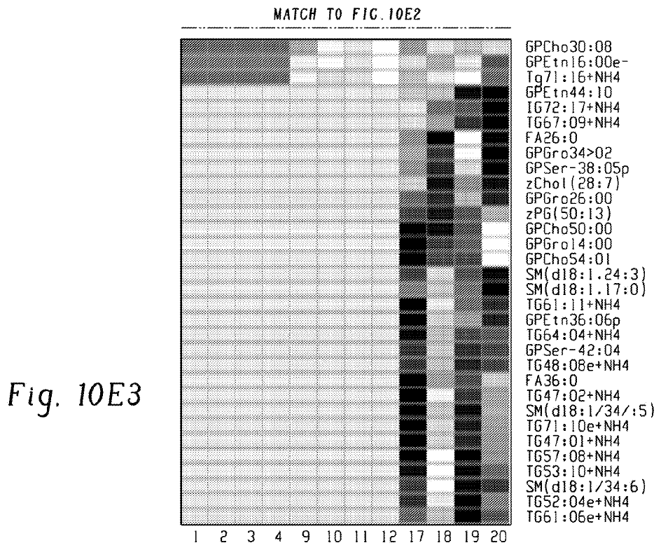

In the partial least squares-discriminant analysis (PLS-DA) score plot, clusters of lipid species from the VLCPUFA groups were observed as separate clusters from those of the control diet group, and 1% and 5% VLCPUFA groups were also clearly distinguished (FIG. 9). The heatmaps of metabolite abundances in plasma and various key tissues, such as the liver, eye, brain and testis, showed that the VLCPUFA groups, especially 5% VLCPUFA, had a specific profile compared with the control group (FIG. 10). The total levels of the lipid species in the liver extract were dose-dependently reduced by VLCPUFA diet in both male and female mice, and the lipid species contributing to these changes were triglycerides and cholesterol (FIGS. 8B and 8C). Dietary VLCPUFA resulted in remodeling of lipids in the plasma, with incorporation of C24:5 n-3, C26:6 n-3 and C28:8 n-3 (FIG. 11).

### Effect of Dietary VLCPUFA on Body Composition

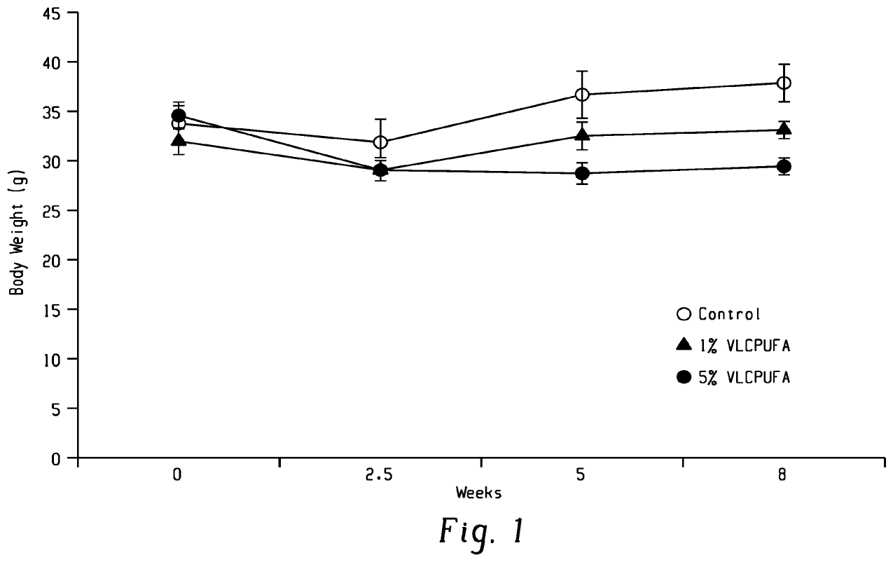

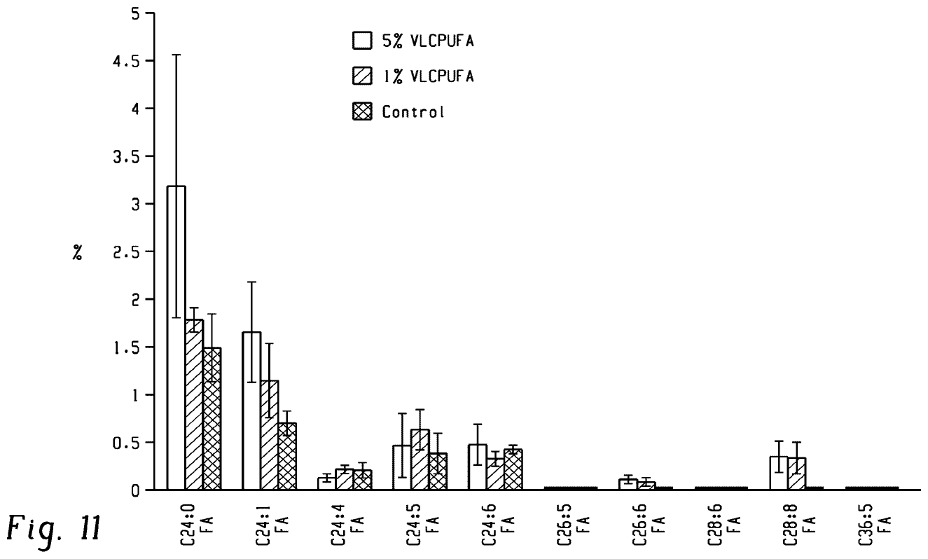

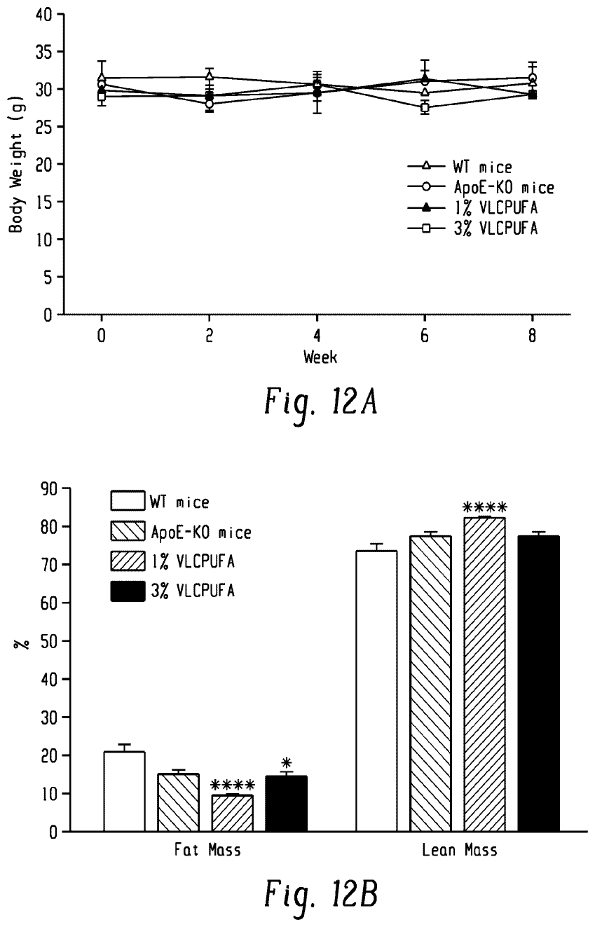

Supplementation with VLCPUFA for 8 weeks tended to decrease body weight in C57BL/6J mice compared with control mice (FIG. 1), and supplementation with 1% VLCPUFA significantly decreased the fat mass and increased the lean mass in ApoE-KO mice compared with the WT mice control, judging by body composition analysis based on NMR (FIG. 12).

### Effect of Dietary VLCPUFA on Atherosclerosis Development

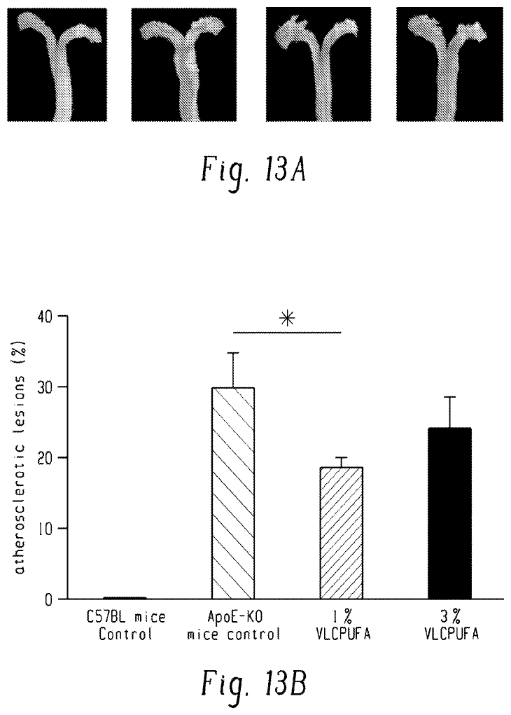

Enface Sudan IV staining of opened aorta section revealed that mice in the 1% VLCPUFA diet group had on average about 30% less atherosclerotic lesion area compared with those in the ApoE-KO mice control group (p<0.05) (FIG. 13).

### Effect of Dietary VLCPUFA on Cognitive Ability

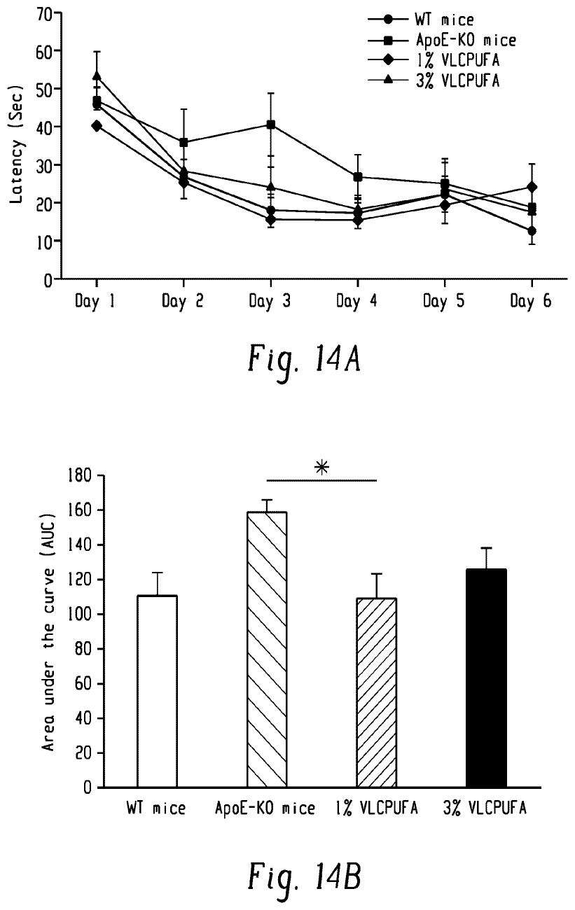

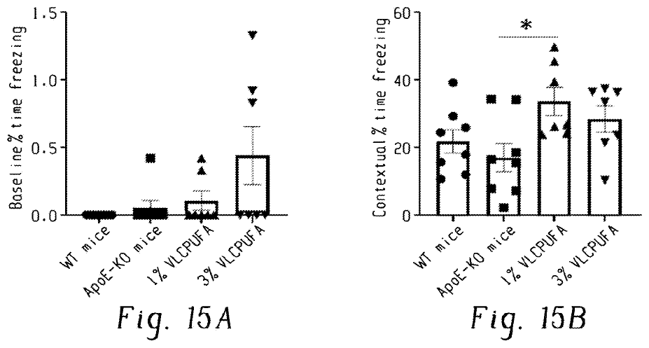

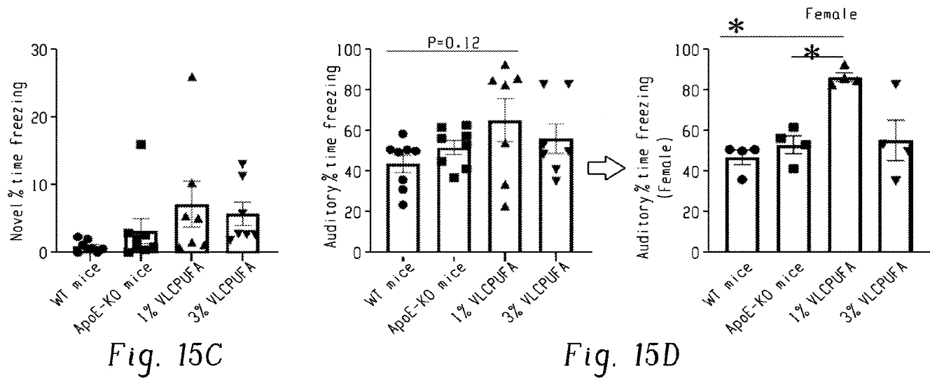

In the Morris water maze test, the mouse relies on visual cues to navigate to a submerged escape platform. Spatial learning was assessed by daily repeated trials for 6 days. The latency to reach the hidden platform for the ApoE-KO mice on VLCPUFA diet was significantly decreased compared with the ApoE-KO mice control (FIG. 14). In the contextual learning test, the baseline % time freezing and the novel context % time freezing were low and no significant differences were observed among the four groups of mice. However, contextual and auditory % time freezing were significantly increased in mice on VLCPUFA diet, suggesting that the dietary VLCPUFA improved spatial learning, memory, and contextual learning in mice (FIG. 15).

Agonist Activity of VLCPUFA with Peroxisome Proliferator-Activated Receptors (PPARs)

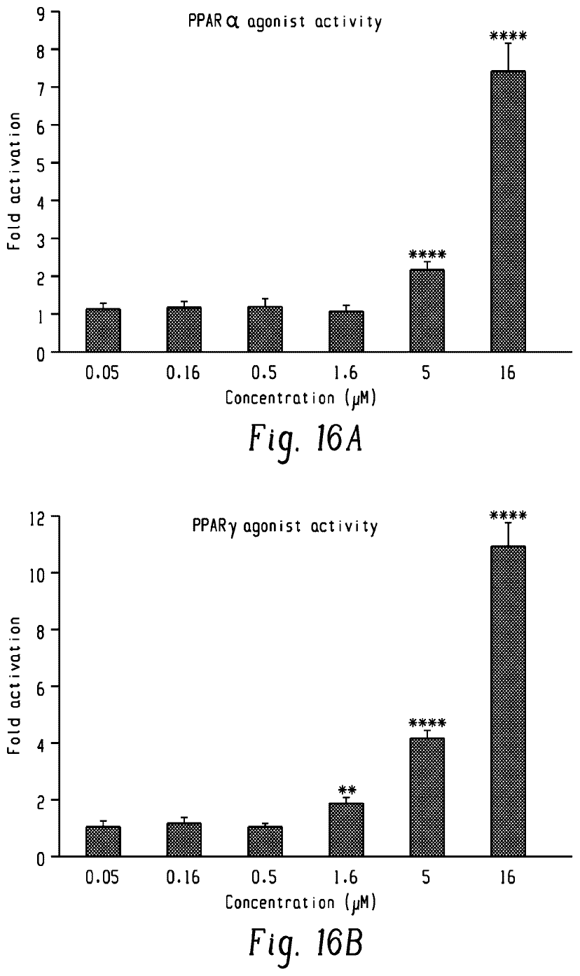

To evaluate VLCPUFA oil for agonist activity with human PPARα and PPARγ receptors, reporter cells were treated with 6 concentrations of VLCPUFA oil, starting at 16 μM and continuing with 3.17-fold serial dilutions. All treatment concentrations were performed in triplicate. The vehicle ethanol did not show agonist activity, but the VLCPUFA oil showed agonist activity with PPARα and PPARγ receptors in a dose-dependent manner (FIG. 16).

TABLE 1
Levels of the major fatty acids in fish
oil-derived VLCPUFA concentrate oil

| Fatty Acid | Level (%)* |
|---|---|
| C20:1 n-9 | 6.6 |
| C20:2 n-6 | 6.4 |
| C20:4 n-6 | 8.5 |
| C20:5 n-3 | 0.4 |
| C22:4 n-6 | 6.1 |
| C22:5 n-3 | 2.0 |
| C22:6 n-3 | 10.1 |
| C24:5 n-3 | 28.5 |
| C26:6 n-3 | 3.3 |
| C28:8 n-3 | 8.5 |

The values correspond to the mean of three separate samples processed independently.
*There are trace amounts of other VLCPUFA isomers, including C24:4 n-3 and C25:5 n-3.

TABLE 2
Plasma clinical biochemistry of C57BL/6J mice
in a 8-week feeding study of VLCPUFA oil

| | Control | 1% VLCPUFA | 5% VLCPUFA |
|---|---|---|---|
| Total bilirubin (mg/dL) | <0.1 | <0.1 | <0.1 |
| Direct bilirubin (mg/dL) | <0.2 | <0.2 | <0.2 |
| Chloride (mmol/L) | <60 | <60 | <60 |
| Calcium (mmol/L) | <0.20 | <0.20 | <0.20 |
| Magnesium (mmol/L) | <0.10 | <0.10 | <0.10 |
| Creatinine (mg/dL) | <0.1 | <0.1 | <0.1 |
| Alkaline phosphatase (U/L) | 12.97 ± 1.95 | 10 ± 1.09 | 8.62 ± 0.75 |
| ALT/GPT (U/L) | 36.56 ± 10.38 | 22.25 ± 2.14 | 37 ± 5.44 |
| AST/GOT (U/L) | 74.59 ± 0.17 | 60.5 ± 0.12 | 67.88 ± 0.07 |
| Potassium (mmol/L) | 3.76 ± 0.17 | 3.39 ± 0.12 | 3.5 ± 0.07 |
| Albumin (g/dL) | 2.14 ± 0.07 | 1.96 ± 0.07 | 2.04 ± 0.04 |
| Total creatine kinase (U/L) | 339.47 ± 107.61 | 418 ± 147.38 | 628.75 ± 95.46 |
| Lactate dihydrogen (U/L) | 260.44 ± 20.27 | 265.37 ± 29.09 | 281 ± 31.54 |
| Total protein (g/dL) | 3.09 ± 0.11 | 2.76 ± 0.1 | 2.84 ± 0.05 |
| Amylase (U/L) | 1546.59 ± 106.63 | 1215.88 ± 85.52 | 1138.25 ± 49.27 |
| Inorganic phosphorus (mg/dL) | 3.22 ± 0.09 | 3.09 ± 0.14 | 3.89 ± 0.26 |
| Urea nitrogen (mg/dL) | 9.47 ± 0.2 | 8.38 ± 0.5*** | 5.87 ± 0.3**** |
| Uric Acid (mg/dL) | 0.46 ± 0.06 | 0.39 ± 0.04 | 0.29 ± 0.02* |

Values are mean ± SEM (n = 8).
*P < 0.05,
***P < 0.001,
****P < 0.0001 significantly different from Control.

TABLE 3
Effects of 2-week repeated gavage of VLCPUFA oil in C57BL/6J mice on retina and
retinal pigment epithelium (RPE) levels of fatty acids with C < 24 chain length

| | Retina | | RPE | |
|---|---|---|---|---|
| FA (%) | Control | VLCPUFA | Control | VLCPUFA |
| 14:0 | 0.04 ± 0.01 | 0.19 ± 0.05* | N.D. | N.D. |
| 15:0 | 0.086 ± 0.003 | 0.092 ± 0.021 | 0.18 ± 0.02 | 0.15 ± 0.03 |
| 16:0 | 23 ± 0.91 | 30.43 ± 0.44** | 17.16 ± 0.46 | 16.35 ± 0.85 |
| 17:0 | 0.33 ± 0.01 | 0.35 ± 0.04 | 0.75 ± 0.03 | 0.79 ± 0.07 |
| 18:0 | 28.8 ± 4.35 | 30.58 ± 0.62 | 23.46 ± 1.13 | 25.44 ± 1.2 |
| 20:0 | 0.68 ± 0.13 | 0.45 ± 0.03 | 2.57 ± 0.4 | 2.99 ± 0.17 |
| 22:0 | 0.26 ± 0.1 | 0.12 ± 0.02 | 3.13 ± 0.07 | 3.2 ± 0.13 |
| 24:0 | 0.088 ± 0.043 | 0.033 ± 0.007 | 2.67 ± 0.07 | 2.71 ± 0.18 |
| SAT FAs | 53.28 ± 4.56 | 62.25 ± 1.03 | 49.93 ± 1.55 | 51.63 ± 2.07 |
| 16:1 | 0.48 ± 0.04 | 0.71 ± 0.03*** | 1.59 ± 0.41 | 0.76 ± 0.06 |
| 18:1 | 7.79 ± 4.73 | 2.94 ± 0.05 | 2.71 ± 0.19 | 2.61 ± 0.16 |
| 20:1 | 0.093 ± 0.033 | 0.014 ± 0.009 | 0.75 ± 0.41 | 1.91 ± 0.66 |
| 24:1 | 0.026 ± 0.01 | 0.012 ± 0.01 | 2.39 ± 0.36 | 3.58 ± 0.49 |
| MUFAs | 8.45 ± 4.72 | 3.7 ± 0.08 | 7.44 ± 1 | 8.86 ± 1.17 |
| 18:2 n-6 | 3.02 ± 1.37 | 1.62 ± 0.12 | 5.64 ± 0.39 | 3.93 ± 0.18** |
| 18:3 n-6 | 0.049 ± 0.01 | 0.058 ± 0.012 | 0.19 ± 0.01 | 0.18 ± 0.01 |
| 20:2 n-6 | 0.26 ± 0.04 | 0.27 ± 0.01 | 1.24 ± 0.4 | 0.41 ± 0.14 |
| 20:3 n-6 | 0.4 ± 0.08 | 0.43 ± 0.02 | 0.64 ± 0.05 | 0.4 ± 0.1 |
| 20:4 n-6 | 5.85 ± 1.12 | 6.03 ± 0.22 | 9.77 ± 0.13 | 9.25 ± 0.24 |
| 22:4 n-6 | N.D. | N.D. | 0.32 ± 0.02 | 0.09 ± 0.04** |
| 22:5 n-6 | 0.064 ± 0.006 | 0.074 ± 0.04 | 2.55 ± 0.24 | 1.06 ± 0.41* |
| n-6 PUFAs | 9.64 ± 0.29 | 8.49 ± 0.29* | 20.35 ± 0.44 | 15.31 ± 0.42**** |
| 18:2 n-3 | 9.22 ± 1.21 | 10.08 ± 0.15 | 14.34 ± 0.54 | 15.08 ± 0.62 |
| 18:3 n-3 | 0.26 ± 0.01 | 0.01 ± 0 | 0.32 ± 0.04 | 0.38 ± 0.08 |
| 20:2 n-3 | 0.43 ± 0.07 | 0.42 ± 0.04 | 0.64 ± 0.14 | 0.93 ± 0.59 |
| 20:5 n-3 | 0.33 ± 0.04 | 0.3 ± 0.02 | 0.59 ± 0.05 | 0.62 ± 0.03 |
| 22:4 n-3 | 0.41 ± 0.03 | 0.36 ± 0.03 | 0.87 ± 0.07 | 0.88 ± 0.05 |
| 22:5 n-3 | 0.76 ± 0.04 | 0.57 ± 0.04 | 2.16 ± 0.28 | 2.88 ± 0.39 |
| 22:6 n-3 | 17.21 ± 0.6 | 13.79 ± 0.68 | 6.37 ± 0.22 | 5.86 ± 0.24 |
| n-3 PUFAs | 28.63 ± 0.72 | 25.54 ± 0.82* | 25.3 ± 0.71 | 26.63 ± 1.32 |
| n-3/n-6 | 2.98 ± 0.11 | 3.02 ± 0.1 | 1.24 ± 0.02 | 1.74 ± 0.07*** |
| PUFA ratio | | | | |

FA = fatty acid; SAT FAs = saturated fatty acids; MUFAs = mono-unsaturated fatty acids; PUFAs = polyunsaturated fatty acids.
Values are mean ± SEM (n = 6). N.D. = not determined.
*P < 0.05,
**P < 0.01,
***P < 0.001,
****P < 0.0001 significantly different from Control.

TABLE 4
Effects of 2-week repeated gavage of VLCPUFA oil in C57BL/6J mice on retina
and retinal pigment epithelium (RPE) levels of VLCPUFAs (C ≥ 24 chain length)

| | Retina | | RPE | |
|---|---|---|---|---|
| VLCPUFA (%) | Control | VLCPUFA | Control | VLCPUFA |
| 24:4 n-6 | 0.02 ± 0.002 | 0.018 ± 0.002 | 0.48 ± 0.02 | 0.4 ± 0.02 |
| 24:5 n-6 | 0.0031 ± 0.001 | 0.0039 ± 0.0006 | 0.006 ± 0.005 | 0.012 ± 0.002 |
| 26:4 n-6 | 0.0009 ± 0.0002 | 0.0017 ± 0.0006 | 0.017 ± 0.001 | 0.012 ± 0.002 |
| 26:5 n-6 | 0.027 ± 0.005 | 0.026 ± 0.004 | 0.029 ± 0.006 | 0.055 ± 0.013 |
| 28:4 n-6 | 0.0007 ± 0.0007 | 0.0034 ± 0.0004** | 0.0017 ± 0.0008 | 0.0029 ± 0.0003 |
| 30:4 n-6 | 0.00018 ± 0.0001 | 0.00013 ± 0.00007 | N.D. | 0.00022 ± 0.0002 |
| 32:5 n-6 | 0.001 ± 0.0003 | 0.0005 ± 0.0001 | 0.0031 ± 0.001 | N.D. |
| 34:4 n-6 | 0.00038 ± 0.0001 | 0.00035 ± 0.0001 | 0.0002 ± 0.0001 | 0.00015 ± 0.0001 |
| n-6 VLCPUFAs | 0.053 ± 0.008 | 0.054 ± 0.007 | 0.53 ± 0.02 | 0.48 ± 0.02 |
| 24:5 n-3 | 0.041 ± 0.003 | 0.035 ± 0.004 | 0.031 ± 0.008 | 0.024 ± 0.001 |
| 24:6 n-3 | 0.12 ± 0.01 | 0.1 ± 0.01 | 0.27 ± 0.03 | 0.2 ± 0.04 |
| 26:5 n-3 | 0.0024 ± 0.0004 | 0.0022 ± 0.0007 | 0.034 ± 0.004 | 0.028 ± 0.004 |
| 26:6 n-3 | 0.0063 ± 0.0007 | 0.0033 ± 0.0013 | 0.14 ± 0.01 | 0.08 ± 0.01 |
| 28:5 n-3 | 0.0004 ± 0.00006 | 0.00031 ± 0 | 0.0017 ± 0.0012 | 0.0071 ± 0.0031 |
| 28:6 n-3 | 0.00074 ± 0.0001 | 0.00046 ± 0.0001 | N.D. | 0.0006 ± 0.0012 |
| 30:5 n-3 | 0.00069 ± 0.00022 | 0.00038 ± 0.0001 | 0.0091 ± 0.0008 | 0.0068 ± 0.0012 |
| 30:6 n-3 | 0.0007 ± 0.00015 | 0.00057 ± 0.0001 | 0.027 ± 0.002 | 0.03 ± 0.007 |
| 32:5 n-3 | 0.0022 ± 0.0005 | 0.0018 ± 0.0003 | 0.0012 ± 0.0004 | 0.0005 ± 0.0002 |
| 32:6 n-3 | 0.0096 ± 0.002 | 0.008 ± 0.001 | 0.0068 ± 0.001 | 0.0071 ± 0.001 |
| 34:5 n-3 | 0.0024 ± 0.0007 | 0.0027 ± 0.0004 | 0.0006 ± 0.0002 | 0.00019 ± 0.0001 |
| 34:6 n-3 | 0.0034 ± 0.001 | 0.0032 ± 0.0004 | 0.0022 ± 0.0006 | 0.0006 ± 0.0002 |
| n-3 VLCPUFAs | 0.19 ± 0.02 | 0.16 ± 0.02 | 0.52 ± 0.02 | 0.38 ± 0.06 |
| n-3/n-6 | 3.92 ± 0.56 | 3.14 ± 0.33 | 0.88 ± 0.071 | 0.69 ± 0.1 |
| VLCPUFA ratio | | | | |
| n-6 C18-C34 | 9.69 ± 0.29 | 8.54 ± 0.29* | 20.88 ± 0.44 | 15.83 ± 0.4**** |
| PUFAs | | | | |
| n-3 C18-C34 | 28.82 ± 0.11 | 25.71 ± 0.83* | 25.76 ± 0.69 | 26.99 ± 1.27 |
| PUFAs | | | | |
| n-3/n-6 C18-C34 | 2.97 ± 0.11 | 3.02 ± 0.1 | 1.23 ± 0.02 | 1.71 ± 0.07*** |
| PUFAs ratio | | | | |

PUFAs = polyunsaturated fatty acids; VLCPUFAs = very long chain polyunsaturated fatty acids.
Values are mean ± SEM (n = 6). N.D. = not determined.
*P < 0.05,
**P < 0.01,
***P < 0.001,
****P < 0.0001 significantly different from Control.

1. A method of treating or decreasing the risk of developing age-related macular degeneration in a subject in need thereof, comprising administering to said subject a therapeutically effective amount of a 24-carbon to 40-carbon very long chain polyunsaturated fatty acid (VLCPUFA).

2. The method of claim 1, wherein the VLCPUFA is C24:5 n-3, C26:6 n-3 or C28:8 n-3, or any combination or all thereof.

3. The method of claim 1, wherein the VLCPUFA is derived from fish oil or made by chemical synthesis.

4. A method of treating, or ameliorating one or more symptoms of, impaired retina function in a subject in need thereof, comprising administering to said subject a therapeutically effective amount of a 24-carbon to 40-carbon very long chain polyunsaturated fatty acid (VLCPUFA).

5. The method of claim 4, wherein the VLCPUFA is C24:5 n-3, C26:6 n-3 or C28:8 n-3, or any combination or all thereof.

6. The method of claim 4, wherein the VLCPUFA is derived from fish oil or made by chemical synthesis.

7. The method of claim 4, wherein the subject has retina ageing.

8. A method of treating hyperlipidemia, hypercholesterolemia or hypertriglyceridemia, or any combination or all thereof in a subject in need thereof, comprising administering to said subject a therapeutically effective amount of a 24-carbon to 40-carbon very long chain polyunsaturated fatty acid (VLCPUFA).

9. The method of claim 8, wherein the VLCPUFA is C24:5 n-3, C26:6 n-3 or C28:8 n-3, or any combination or all thereof.

10. The method of claim 8, wherein the VLCPUFA is derived from fish oil or made by chemical synthesis.

11. A method of treating metabolic syndrome, obesity or overweight in a subject in need thereof, comprising administering to said subject a therapeutically effective amount of a 24-carbon to 40-carbon very long chain polyunsaturated fatty acid (VLCPUFA).

12. The method of claim 11, wherein the VLCPUFA is C24:5 n-3, C26:6 n-3 or C28:8 n-3, or any combination or all thereof.

13. The method of claim 11, wherein the VLCPUFA is derived from fish oil or made by chemical synthesis.

14. A method of treating hepatic steatosis or fatty liver disease in a subject in need thereof, comprising administering to said subject a therapeutically effective amount of a 24-carbon to 40-carbon very long chain polyunsaturated fatty acid (VLCPUFA).

15. The method of claim 14, wherein the VLCPUFA is C24:5 n-3, C26:6 n-3 or C28:8 n-3, or any combination or all thereof.

16. The method of claim 14, wherein the VLCPUFA is derived from fish oil or made by chemical synthesis.

17. A method of treating an elevated glucose concentration in blood, plasma or serum, or impaired fasting glucose, postprandial hyperglycemia, impaired glucose tolerance, insulin resistance or diabetes, in a subject in need thereof, comprising administering to said subject a therapeutically effective amount of a 24-carbon to 40-carbon very long chain polyunsaturated fatty acid (VLCPUFA).

18. The method of claim 17, wherein the VLCPUFA is C24:5 n-3, C26:6 n-3 or C28:8 n-3, or any combination or all thereof.

19. The method of claim 17, wherein the VLCPUFA is derived from fish oil or made by chemical synthesis.

20. A method of treating, or ameliorating one or more symptoms of, atherosclerosis in a subject in need thereof, comprising administering to said subject a therapeutically effective amount of a 24-carbon to 40-carbon very long chain polyunsaturated fatty acid (VLCPUFA).

21. The method of claim 20, wherein the VLCPUFA is C24:5 n-3, C26:6 n-3 or C28:8 n-3, or any combination or all thereof.

22. The method of claim 20, wherein the VLCPUFA is derived from fish oil or made by chemical synthesis.

23. A method of treating, or ameliorating one or more symptoms of, an adipofascial disorder in a subject in need thereof, comprising administering to said subject a therapeutically effective amount of a 24-carbon to 40-carbon very long chain polyunsaturated fatty acid (VLCPUFA).

24. The method of claim 23, wherein the VLCPUFA is C24:5 n-3, C26:6 n-3 or C28:8 n-3, or any combination or all thereof.

25. The method of claim 23, wherein the VLCPUFA is derived from fish oil or made by chemical synthesis.

26. A method of treating, or ameliorating one or more symptoms of, impaired cognitive function in a subject in need thereof, comprising administering to said subject a therapeutically effective amount of a 24-carbon to 40-carbon very long chain polyunsaturated fatty acid (VLCPUFA).

27. The method of claim 26, wherein the VLCPUFA is C24:5 n-3, C26:6 n-3 or C28:8 n-3, or any combination or all thereof.

28. The method of claim 26, wherein the VLCPUFA is derived from fish oil or made by chemical synthesis.

29. A method of activating a peroxisome proliferator-activated receptor (PPAR)-alpha pathway in a subject, comprising administering to said subject a therapeutically effective amount of a 24-carbon to 40-carbon very long chain polyunsaturated fatty acid (VLCPUFA).

30. The method of claim 29, wherein the VLCPUFA is C24:5 n-3, C26:6 n-3 or C28:8 n-3, or any combination or all thereof.

31. A method of activating a peroxisome proliferator-activated receptor (PPAR)-gamma pathway in a subject, comprising administering to said subject a therapeutically effective amount of a 24-carbon to 40-carbon very long chain polyunsaturated fatty acid (VLCPUFA).

32. The method of claim 31, wherein the VLCPUFA is C24:5 n-3, C26:6 n-3 C28:8 n-3, or any combination or all thereof.
